(19) United States
(12) Patent Application Publication
(10) Pub. No.: US 2025/0230471 A1
(43) Pub. Date: Jul. 17, 2025

(54) GENOME INSERTIONS IN CELLS

(71) Applicant: Addition Therapeutics, Inc., South San Francisco, CA (US)

(72) Inventors: Reema ALDAIMALANI, Berkeley, CA (US); Molly BROTHERS, Oakland, CA (US); Miguel GARCIA, El Cerrito, CA (US); Robert KLEIN, Palo Alto, CA (US); Heather UPTON, Oakland, CA (US); Yan WANG, San Francisco, CA (US)

(21) Appl. No.: 19/026,058

(22) Filed: Jan. 16, 2025

Related U.S. Application Data
(63) Continuation of application No. PCT/US2023/028175, filed on Jul. 19, 2023.
(60) Provisional application No. 63/390,863, filed on Jul. 20, 2022.

(51) Int. Cl. C12N 15/90; C12N 9/12; C12N 15/113; C12N 15/88

(52) U.S. Cl. CPC C12N 15/907; C12N 9/1276; C12N 15/113; C12N 15/88; C12Y 207/07049; C12N 2310/12

(57) ABSTRACT

The present disclosure provides compositions and methods for inserting heterologous payload sequences into a target-site in a host cell genome. The compositions and methods use non-LTR retrotransposon reverse transcriptase proteins that bind template RNAs comprising a payload sequence that encodes a protein or regulatory RNA. The template RNA can comprise modified uridines that are not cleavable by a ribozyme. The incorporation of modified uridines increases the efficiency of integration and expression of the payload sequence and decreases cellular toxicity.

### CROSS-REFERENCE TO RELATED APPLICATIONS

This application is a continuation of PCT/US2023/028175, filed Jul. 19, 2023, which claims priority to U.S. Provisional Patent Application No. 63/390,863, filed Jul. 20, 2022, each of which is incorporated by reference in its entirety for all purposes.

### SEQUENCE LISTING

The instant application contains a Sequence Listing which has been submitted electronically in XML format and is hereby incorporated by reference in its entirety. Said XML copy, created on Jan. 16, 2025, is named 67098-702.301_SL.xml and is 52,891 bytes in size.

### BACKGROUND OF THE INVENTION

Insertion of DNA transgenes into the genomic DNA of an organism is associated with several undesirable side effects. For example, introducing DNA into the cytoplasm of a cell can induce an immune response that can be harmful to cells or the organism. In addition, current methods for integration of DNA at a target site in the host cell genome via homologous recombination requires introduction of a potentially mutagenic double-strand break in the genomic DNA. Further, DNA integration in post-mitotic cells such as neurons can occur at non-specific locations due to the fact that homologous recombination occurs more efficiently in dividing cells.

The present disclosure provides compositions and methods that improve gene editing at target sites in a host cell genome. The methods can be used for gene therapy applications and provide advantages over current DNA-based and viral vector-based gene therapy methods.

### BRIEF SUMMARY OF THE INVENTION

The instant disclosure provides compositions and methods that improve gene therapy technologies for introducing heterologous polynucleotides into a target cell.

In one aspect, the disclosure provides a method of inserting a heterologous polynucleotide at a target site in a eukaryotic genome, the method comprising transfecting a eukaryotic cell with: (a) an RNA encoding a non-LTR retrotransposon reverse transcriptase protein (nrRT) comprising a reverse transcriptase domain and an endonuclease domain; and (b) a template RNA. In some embodiments, the template RNA comprises a promoter, a payload sequence, a polyA sequence, and a nrRT binding sequence. In some embodiments, the template RNA comprises one or more modified uridine (U) nucleosides selected from the group consisting of N1-methyl-pseudouridine (N1mΨU), pseudouridine (ΨU), 5-methyluridine (5meU), 5-methyoxyuridine (5moU), and mixtures thereof. In some embodiments, the template RNA comprises a mixture comprising unmodified uridines and one or more modified uridines selected from the group consisting of N1mΨU, ΨU, 5meU, and 5moU. In some embodiments, the template RNA comprising modified uridines is not cleavable by a ribozyme.

In some embodiments, the nrRT is expressed in the cell and catalyzes insertion of a double stranded heterologous polynucleotide comprising the payload sequence at the target site in the eukaryotic genome.

In some embodiments, the template RNA comprising a modified U increases the insertion efficiency of the payload sequence into the eukaryotic genome compared to a template RNA comprising an unmodified U.

In some embodiments, the template RNA further comprises a 5′ ribozyme sequence selected from an active ribozyme, a partially active ribozyme, a ribozyme having reduced catalytic activity, or a catalytically inactive ribozyme. In some embodiments, the 5′ ribozyme is selected from an HDV ribozyme, a TriCasA ribozyme, or a native cognate ribozyme, a semi-cognate ribozyme, or variants thereof. In some embodiments, the 5′ ribozyme sequence comprises a sequence selected from any one of SEQ ID NOs: 3 or 13 to 22 (without the pp7 binding sequence), or a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 3 or 13-22 (without the pp7 binding sequence).

In some embodiments, the template RNA does not comprise a functional 5′ ribozyme sequence or does not comprise a 5′ ribozyme sequence.

In some embodiments, cellular toxicity is decreased when the template RNA comprises a modified U.

In some embodiments, the template RNA further comprises a 5′ sequence that protects the 5′ end from degradation.

In some embodiments, the template RNA further comprises a 5′ sequence that promotes site-specific insertion of the heterologous polynucleotide into a target site in the eukaryotic genome.

In some embodiments, wherein the nrRT binding sequence comprises a 3′UTR sequence. In some embodiments, the 3′UTR sequence is isolated from an organism selected from the group consisting of G. aculeatus, D. melanogaster, L. polyphemus, P. pungitis, N. vitripennis, G. fortis, O. latipes, Z. albicollis, T. guttata, T. castaneum, T. guttatus, D. simulans, B. mori, and A. vaga,. In some embodiments, the 3′UTR comprises a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 26-39.

In some embodiments, the template RNA further comprises a 3′ sequence that promotes site-specific insertion of the heterologous polynucleotide into the eukaryotic genome, and/or enhances the efficiency and fidelity of target-primed reverse transcription.

In some embodiments, the template RNA further comprises one or more of i) an RNA polymerase terminator, ii) a sequence useful for purification, iii) a sequence encoding a protein that is useful for enrichment, iv) a Kozak sequence 5′ of the payload sequence, and/or v) a polyA sequence located 3′ of the nrRT binding sequence.

In some embodiments, the template RNA further comprises a) a 5′ sequence that is homologous to a DNA sequence located 5′ to a target insertion site in the eukaryotic genome; or (b) a 3′ sequence that is homologous to a DNA sequence located 3′ to a target insertion site in the eukaryotic genome; or both (a) and (b).

In some embodiments, the template RNA lacks a 5′ phosphate.

In some embodiments, the payload sequence encodes a therapeutic protein that replaces or complements a defective gene or protein. In some embodiments, the therapeutic protein is selected from the group consisting of Factor VIII, Factor IX, and phenylalanine hydroxylase (PAH).

In some embodiments, the payload sequence encodes an inhibitor of another protein. In some embodiments, the inhibitor is single chain antibody.

In some embodiments, the payload sequence encodes a regulatory RNA.

In some embodiments, wherein the payload sequence encodes a protein selected from a gene in Table 7.

In some embodiments, modulating i) the molar ratio of the nrRT mRNA to the template RNA and/or ii) the amount of total RNA delivered to the target cell increases the insertion efficiency.

In some embodiments, the RNA encoding the nrRT comprises one or more modified uridine (U) nucleosides selected from the group consisting of N1-methyl-pseudouridine (N1mΨPU), pseudouridine (ΨU), 5-methyluridine (5meU), 5-methyoxyuridine (5moU), and mixtures thereof. In some embodiments, the RNA encoding the nrRT comprises a mixture of unmodified uridines and a modified U selected from the group consisting of N1mΨU, ΨU, 5meU, and 5moU.

In some embodiments, the eukaryotic cell is transfected in vitro. In some embodiments, the eukaryotic cell is transfected in vivo. In some embodiments, the eukaryotic cell is a mammalian cell. In some embodiments, the eukaryotic cell is a human cell. In some embodiments, the human cell is removed from a human subject, transfected (e.g., ex vivo) with the RNA of (a) and (b) to insert the heterologous polynucleotide into the human cell genome, and administered to the human subject.

In some embodiments, the cell is transfected with a LNP formulation, a lipofection reagent, or by electroporation.

In another aspect, the disclosure provides a composition comprising (a) an RNA encoding a non-LTR retrotransposon reverse transcriptase protein (nrRT) comprising a reverse transcriptase domain and an endonuclease domain; and (b) a template RNA. In some embodiments, the template RNA comprises a promoter, a payload sequence, a polyA sequence, and a nrRT binding sequence. In some embodiments, the template RNA comprises one or more modified uridine (U) nucleosides selected from the group consisting of N1-methyl-pseudouridine (N1mΨU), pseudouridine (ΨU), 5-methyluridine (5meU), 5-methyoxyuridine (5moU), and mixtures thereof. In some embodiments, the template RNA comprises a mixture comprising unmodified uridines and one or more modified uridines selected from the group consisting of N1mΨU, ΨU, 5meU, and 5moU. In some embodiments, the template RNA comprising modified uridines is not cleavable by a ribozyme.

In some embodiments, the template RNA further comprises a 5′ ribozyme sequence selected from an active ribozyme, a partially active ribozyme, a ribozyme having reduced catalytic activity, or a catalytically inactive ribozyme. In some embodiments, the 5′ ribozyme is selected from an HDV ribozyme, a TriCasA ribozyme, or a native cognate ribozyme, a semi-cognate ribozyme, or variants thereof. In some embodiments, the 5′ ribozyme sequence comprises a sequence selected from any one of SEQ ID NOs: 3 or 13 to 22 (without the pp7 binding sequence), or a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 3 or 13-22 (without the pp7 binding sequence).

In some embodiments, the template RNA does not comprise a functional 5′ ribozyme sequence or does not comprise a 5′ ribozyme sequence.

In some embodiments, the template RNA further comprises a 5′ sequence that protects the 5′ end from degradation.

In some embodiments, the template RNA further comprises a 5′ sequence that promotes site-specific insertion of the heterologous polynucleotide into a target site in the eukaryotic genome.

In some embodiments, wherein the nrRT binding sequence comprises a 3′UTR sequence. In some embodiments, the 3′UTR sequence is isolated from an organism selected from the group consisting of G. aculeatus, D. melanogaster, L. polyphemus, P. pungitis, N. vitripennis, G. fortis, 0. latipes, Z. albicollis, T. guttata, T. castaneum, T. guttatus, D. simulans, B. mori, and A. vaga,. In some embodiments, the 3′UTR comprises a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 26-39.

In some embodiments, the template RNA further comprises a 3′ sequence that promotes site-specific insertion of the heterologous polynucleotide into the eukaryotic genome, and/or enhances the efficiency and fidelity of target-primed reverse transcription.

In some embodiments, the template RNA further comprises one or more of i) an RNA polymerase terminator, ii) a sequence useful for purification, iii) a sequence encoding a protein that is useful for enrichment, iv) a Kozak sequence 5′ of the payload sequence, and/or v) a polyA sequence located 3′ of the nrRT binding sequence.

In some embodiments, the template RNA further comprises a) a 5′ sequence that is homologous to a DNA sequence located 5′ to a target insertion site in the eukaryotic genome; or (b) a 3′ sequence that is homologous to a DNA sequence located 3′ to a target insertion site in the eukaryotic genome; or both (a) and (b).

In some embodiments, the template RNA lacks a 5′ phosphate.

In some embodiments, the payload sequence encodes a therapeutic protein that replaces or complements a defective gene or protein. In some embodiments, the therapeutic protein is selected from the group consisting of Factor VIII, Factor IX, and phenylalanine hydroxylase (PAH).

In some embodiments, the payload sequence encodes an inhibitor of another protein. In some embodiments, the inhibitor is single chain antibody.

In some embodiments, the payload sequence encodes a regulatory RNA.

In some embodiments, wherein the payload sequence encodes a protein selected from a gene in Table 7.

In some embodiments, the RNA encoding the nrRT comprises one or more modified uridine (U) nucleosides selected from the group consisting of N1-methyl-pseudouridine (N1mΨU), pseudouridine (ΨU), 5-methyluridine (5meU), 5-methyoxyuridine (5moU), and mixtures thereof. In some embodiments, the RNA encoding the nrRT comprises a mixture of unmodified uridines and a modified U selected from the group consisting of N1mΨU, ΨU, 5meU, and 5moU.

In another aspect, the disclosure provides a pharmaceutical composition. The pharmaceutical composition can comprise a composition described herein. In some embodiments, the pharmaceutical composition is formulated in a lipid nanoformulation selected from a liposome or a lipid nanoparticle (LNP). In some embodiments, the pharmaceutical composition further comprises a pharmaceutically acceptable excipient or salt.

In another aspect, the disclosure provides a method of treating a disease or condition in a subject in need if treatment. In some embodiments, the method comprises administering an effective amount of a pharmaceutical composition of the disclosure to the subject.

In some embodiments, the disease or condition is selected from the group consisting of Sickle cell anemia, Severe Combined Immunodeficiency (ADA-SCID/X-SCID), Cystic fibrosis, Hemophilia, Duchenne muscular dystrophy, Huntington's disease, Parkinson's, Hypercholesterolemia, Alpha-1 antitrypsin, Chronic granulomatous disease, Fanconi Anemia and Gaucher Disease. In some embodiments, wherein the disease or condition is selected from Table 7.

### BRIEF DESCRIPTION OF THE DRAWINGS

FIG. 1 is a diagram showing delivery of the two RNA compositions of the disclosure into the cytoplasm of a target cell (left panel) and the proposed mechanism of action of insertion of a heterologous polynucleotide into the genomic DNA of the target cell (right panel).

FIG. 2 shows a diagram of an exemplary mRNA encoding an nrRT, an exemplary template RNA, and an exemplary delivery formulation of the disclosure.

FIG. 3 shows the structure of uridine and modified uridines incorporated into RNAs of the disclosure.

FIG. 4 shows that incorporation of a modified uridine into the template RNA results in successful integration of the payload sequence into the host cell genome. Template RNA comprising the modified uridine 5meU and a payload sequence encoding GFP was cleaved by the 5′ ribozyme HDV-gu6 (left panel). Transfected cells expressed GFP (right panel).

FIG. 5 shows incorporation of the modified uridine N1-methyl-pseudouridine (N1mΨU) into the template RNA was not cleaved by the HDV_gu6 ribozyme (left panel), but the payload sequence encoding GFP was still successfully integrated into the host cell genome (right panel).

FIG. 6 shows expression of the payload sequence encoding GFP in cells transfected with different template RNAs incorporating different modified uridines. The results demonstrate that the modified uridines N1-methyl-pseudouridine (N1mΨU) and pseudouridine (ΨU) produced the highest number of GFP positive cells and lowest toxicity, even though these template RNAs were not cleaved by the 5′ ribozyme (see FIG. 4, left panel).

FIG. 7 shows expression of the payload sequence encoding GFP in cells transfected with template RNAs incorporating N1-methyl-pseudouridine (N1mΨU) and comprising different 5′ modules. The results demonstrate that template RNAs comprising catalytically inactive ribozymes (HDV_gu5b_CatDead) and template RNAs with the ribozyme sequence deleted (SL28, 28noRZ) still resulted in successful integrated of the payload sequence into the host cell genome. The “+” and “−” indicate the presence or absence of the indicated structure (RZ Seq.; RZ fold) or activity (RZ Act.) for each ribozyme. For activity (RZ Act.), the “-” indicates that the ribozymes sequence did not cleave the indicated nucleotide substitutions.

### DEFINITIONS

Unless defined otherwise, all technical and scientific terms used herein have the same meaning as commonly understood by one of ordinary skill in the art to which this invention pertains. Although essentially any methods and materials similar to those described herein can be used in the practice or testing of the present invention, only exemplary methods and materials are described. For purposes of the present invention, the following terms are defined below.

The terms “a,” “an,” and “the” include plural referents, unless the context clearly indicates otherwise.

The term “cognate” as used herein refers to an nrRT protein and a template RNA, where the nrRT protein preferentially binds a specific template RNA. The nrRT protein and its cognate template RNA may occur in nature (referred to as native protein and template), or one or both of the nrRT protein and template RNA may be modified to preferentially bind to another nrRT protein and/or template RNA.

The term “native” refers to a nucleic acid or protein found in nature or in its natural configuration when present in another organism or cell.

The term “ribozyme” refers to an RNA molecule having enzymatic activity. The term includes self-cleaving ribozymes that catalyze sequence-specific intramolecular cleavage of RNA, including cleavage in cis (on the same strand).

The term “native ribozyme” refers to a ribozyme found in nature, e.g., a wild-type ribozyme, and includes different ribozymes found in different organisms.

The term “cognate ribozyme” refers to a ribozyme sequence that preferentially associates with a native or naturally occurring nrRT protein.

The term “semi-cognate” ribozyme refers to a ribozyme from a closely related species that associates with a nrRT protein.

The term “HDV RZ fold” refers to an RNA sequence that comprises the fold of the hepatitis delta virus (HDV) ribozyme and which retains ribozyme function.

The term “non-LTR retrotransposon reverse transcriptase protein” or “nrRT protein” refers to a reverse transcriptase protein that can copy a template RNA into cDNA at a target site in the host cell genome, where cDNA synthesis is primed by a nick introduced by the nrRT protein at the target-site, which leads to stable, double-stranded transgene insertion. The term also includes modified variants of an nrRT protein having increased efficiency or modified nicking activity or modified binding properties (affinity) to a template RNA.

The term “template RNA” refers to a single stranded RNA that binds to a nrRT protein and serves as a template for first strand cDNA synthesis at a target-site in the host cell genome.

The term “payload” refers to a compound, protein, inhibitor, or nucleic acid that is inserted into the genome of a host cell using the compositions and methods of the disclosure.

The term “encode,” “encodes” or “encoding” refers to transcription and/or translation of an RNA sequence to produce a product. The product can be a polypeptide, protein, or functional RNA.

The term “operably linked” refers to a sequence that is joined in a functional relationship with another sequence. For example, a promoter or enhancer is operably linked to a payload sequence if it modulates the transcription of the sequence. The term includes nucleic acid sequences that are covalently linked in a plasmid or vector, regardless of the number of nucleotides in between the sequences. For example, a promoter is operably linked to a polyA sequence even if a payload sequence is present between the promoter and polyA sequence.

The term “junction” refers to the location in a host cell genome where the genomic DNA is connected to the inserted double stranded cDNA.

The term “lipid nanoparticle” or “LNP” refers to a delivery vehicle comprising one or more lipids (e.g., cationic lipids, non-cationic lipids, PEG-modified lipids).

The term “liposome” generally refers to a vesicle composed of lipids (e.g., amphiphilic lipids) arranged in one or more spherical bilayers or bilayers.

As used herein, “percentage of sequence identity” is determined by comparing two optimally aligned sequences over a comparison window, wherein the portion of the sequence in the comparison window can comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or “identity,” in the context of two or more nucleic acids or polypeptide sequences, refer to two or more sequences or subsequences that are the same. Sequences are “substantially identical” to each other if they have a specified percentage of nucleotides or amino acid residues that are the same (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% identity over a specified region), when compared and aligned for maximum correspondence over a comparison window, or designated region as measured using one of the following sequence comparison algorithms or by manual alignment and visual inspection. These definitions also refer to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters are commonly used, or alternative parameters can be designated. The sequence comparison algorithm then calculates the percent sequence identities or similarities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4.

The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “heterologous” refers to any polynucleotide or polypeptide sequence that is not naturally occurring in a host cell or organism or is inserted in a location not naturally occurring in the host cell or organism.

The term “vector” refers to DNA, typically double-stranded DNA, which comprises foreign or heterologous DNA. The term includes plasmids and viral vectors. Vectors can contain polynucleotide sequences that facilitate the autonomous replication of the vector in a host cell. The vector can be used to replicate the foreign or heterologous DNA in a suitable host cell. In addition, the vector can also contain elements that permit transcription of the inserted DNA into one or more mRNA molecules. Expression vectors additionally contain sequence elements operably linked the inserted DNA that increase the half-life of the expressed mRNA and/or allow translation of the mRNA into a protein molecule.

### DETAILED DESCRIPTION OF THE INVENTION

The instant disclosure provides compositions and methods that improve gene therapy technologies for introducing heterologous polynucleotides into a target cell. The disclosure provides methods for inserting a heterologous polynucleotide at a target site (site-specific integration) in the genome of a target cell. The heterologous polynucleotide can comprise a transgene encoding a therapeutic protein or a non-protein regulator element. In some embodiments, the cell is a eukaryotic cell, such as a mammalian cell.

The instant disclosure provides the numerous advantages over current gene therapy technologies, including: 1) the technology is an RNA-based therapy using RNA-templated gene synthesis into the target cell genome, thereby avoiding problems with DNA delivery into cells such as unintended genetic alterations that can compromise cell function and promote oncogenesis; 2) the heterologous polynucleotide can be inserted into so-called “safe harbor” sites that do not cause deleterious or undesirable alterations to the target cell genome or cellular physiology; 3) there are no known limits on the size or length of the heterologous polynucleotide inserted into the target cell genome; and 4) there is no requirement for cell division, such that post-mitotic cells, such as neurons, can be targeted. Some of the advantages of the instant disclosure, referred to as THERAPEUTIC ADDITION by CONTROLLED SYNTHESIS INSERTION (TASCI™). are shown in Table 1.

TABLE 1
TACSI SOLVES KEY GENE THERAPY PROBLEMS

| | | | DNA | DNA | CRISPR | CRISPR | |
|---|---|---|---|---|---|---|---|
| | | mRNALNP | Viral | LNP | Viral | LNP | |
| | | therapy | therapy | therapy | therapy | therapy | TACSI |
| Efficacy | Long durability of | | ✓ | ✓ | ✓ | ✓ | ✓ |
| | expression | | | | | | |
| | Large insert size | ✓ | | ✓ | | ✓ | ✓ |
| | Full expression | ✓ | ✓* | ✓* | | | ✓ |
| | cassette | | | | | | |
| | Repeat dosing | ✓ | | ✓ | | ✓ | ✓ |
| | Insert full gene in | | ✓* | ✓* | | | ✓ |
| | non-dividing cells | | | | | | |
| Safety | Low mutation | ✓ | | | | | ✓ |
| | potational | | | | | | |
| | Low | ✓ | | ✓ | | ✓ | ✓ |
| | immunigenicity | | | | | | |
| CMC | Simple GMP | ✓ | | ✓ | | ✓ | ✓ |
| | manufacturing | | | | | | |
| | Low COGS | ✓ | | ✓ | | ✓ | ✓ |

*Very high mutagenesis potential.

The compositions and methods of the instant disclosure make use of a two-RNA delivery system for introducing a heterologous polynucleotide into a target cell; 1) a first RNA (e.g., an mRNA) encoding a non-LTR retrotransposon reverse transcriptase protein (nrRT); and 2) a second RNA (also referred to as a template RNA) that comprises a protein coding sequence (or Open Reading Frame “ORF”) and a sequence that binds to the nrRT. The system can further comprise a delivery system for introducing the two RNAs into the cytoplasm of a target cell. In some embodiments, the delivery system comprises a lipid nanoparticle (LNP).

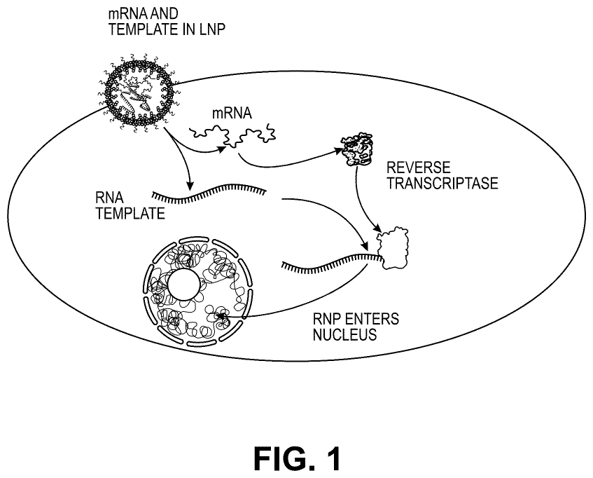

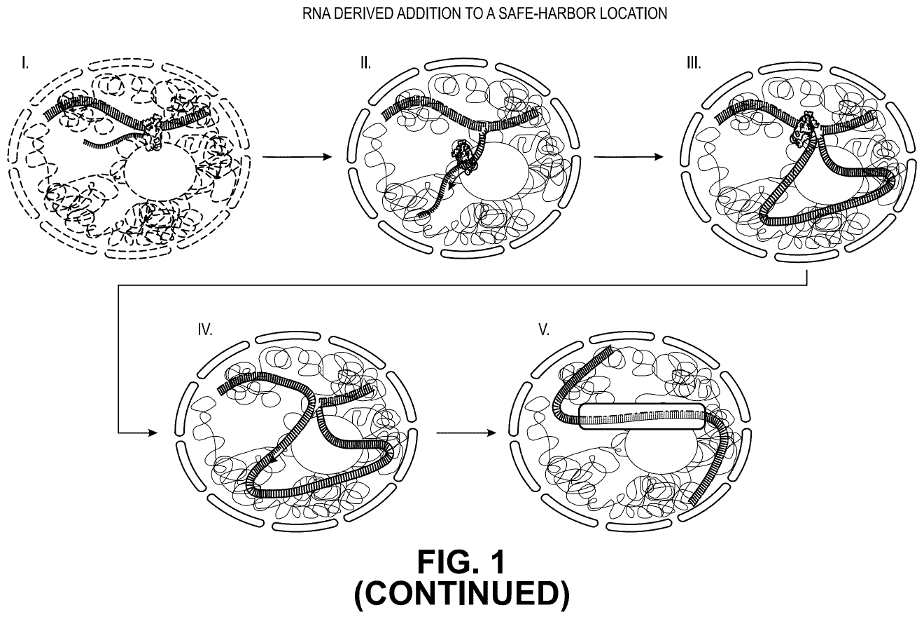

After delivery of the two RNAs into the cytoplasm of the target cell, the mRNA is translated by the endogenous protein synthesis components of the cell to produce the nrRT protein. The nrRT protein then binds to the template RNA, forming a ribonucleoprotein (RNP) complex that enters the nucleus of the target cell. Without being bound by theory, following delivery to the nucleus, it is currently thought that the endonuclease (EN) domain of the nrRT protein cleaves the bottom strand of the target genomic DNA, which provides a 3′ hydroxyl end that serves as a primer for reverse transcription of the template RNA by the reverse transcriptase (RT) domain of the nrRT protein. Following first strand synthesis to produce cDNA, the EN domain or a host endonuclease cleaves the opposite (e.g., the top strand) of the genomic DNA. The nick in the top strand produces another 3′ hydroxyl end that serves as a primer for second strand cDNA synthesis. It is currently unknown if second strand DNA synthesis is performed by the nrRT or by a cellular polymerase. The nick is then repaired, resulting in integration of the double-stranded cDNA into the target site in the genomic DNA. The proposed mechanism is shown in FIG. 1.

It will be understood to a person of skill in the art that the two RNAs do not necessarily comprise an nrRT protein and its naturally occurring cognate template RNA or a modified variant thereof, but that both the nrRT protein and the template RNA can be separately engineered to bind to different nrRT and/or template RNAs.

Eukaryotic Non-LTR Retrotransposon Reverse Transcriptase Protein (nrRT)

In some embodiments, the disclosure provides an RNA (e.g., an mRNA) that encodes an nrRT protein. In some embodiments, the nrRT protein comprises one or more of a DNA binding domain, an RNA biding domain, a reverse transcriptase domain and an endonuclease domain, or combinations thereof. The endonuclease domain of the nrRT proteins of the disclosure produce a single strand nick in the genomic DNA at the target site, producing a free 3′ end of the genomic DNA which serves as a primer for reverse transcription of the template RNA into cDNA. Following first strand cDNA synthesis, the nrRT protein introduces a nick in the second strand, which creates another 3′ end of the genomic DNA that serves as a primer for second strand synthesis of the cDNA at the target site. This results in a double stranded DNA molecule being inserted at the target site in the host cell genomic DNA.

It will be understood that the disclosure encompasses any eukaryotic nrRT protein that can bind and reverse transcribe a template RNA at a target site in the host cell genome. In some embodiments, the nrRT protein comprises an nrRT protein isolated from Zonotrichia albicollis, Taeniopygia guttata, Tinamus guttatus, Geospiza fortis, Pungitis pungitis, Oryzias latipes, Danio rerio, Oryzias melastigma, Petromyzon marinus, Salmo trutta, Salmo salar, Gasterosteus aculeatus, Drosophila mercatorum, Drosophila melanogaster, Nasonia vitripennis, Tribolium castaneum, Drosophila simulans, Apis cerana, Bombyx mori, Lepidurus couesii, Triops cancriformis, Limulus polyphemus, Hydra magnipapillata, Adineta vaga, or Ciona intestinalis, or a modified functional variant thereof. In some embodiments, the mRNA encodes an amino acid sequence that is substantially identical to an nrRT protein isolated from Zonotrichia albicollis, Taeniopygia guttata, Tinamus guttatus, Geospiza fortis, Pungitis pungitis, Oryzias latipes, Danio rerio, Oryzias melastigma, Petromyzon marinus, Salmo trutta, Salmo salar, Gasterosteus aculeatus, Drosophila mercatorum, Drosophila melanogaster, Nasonia vitripennis, Tribolium castaneum, Drosophila simulans, Apis cerana, Bombyx mori, Lepidurus couesii, Triops cancriformis, Limulus polyphemus, Hydra magnipapillata, Adineta vaga, or Ciona intestinalis. In some embodiments, the mRNA encodes an amino acid sequence having at least 60% sequence identity (e.g., greater than or equal to 60%, 65%, 70%, 75%, 80%, 85%, 90%, or 95% identity) to an nrRT protein isolated from Zonotrichia albicollis, Taeniopygia guttata, Tinamus guttatus, Geospiza fortis, Pungitis pungitis, Oryzias latipes, Daniorerio, Oryzias melastigma, Petromyzon marinus, Salmo trutta, Salmo salar, Gasterosteus aculeatus, Drosophila mercatorum, Drosophila melanogaster, Nasonia vitripennis, Tribolium castaneum, Drosophila simulans, Apis cerana, Bombyx mori, Lepidurus couesii, Triops cancriformis, Limulus polyphemus, Hydra magnipapillata, Adineta vaga, or Ciona intestinalis. In some embodiments, the nrRT protein comprises an nrRT protein isolated from other animals.

In some embodiments, the RNA encoding the nrRT comprises one or more of a 5′ cap, a 5′ UTR, an open reading frame (ORF) encoding the nrRT, a 3′ URT, or a polyA sequence at the 3′ end.

In some embodiments, the RNA encoding the nrRT comprises one or more modified uridine (U) nucleosides as described herein.

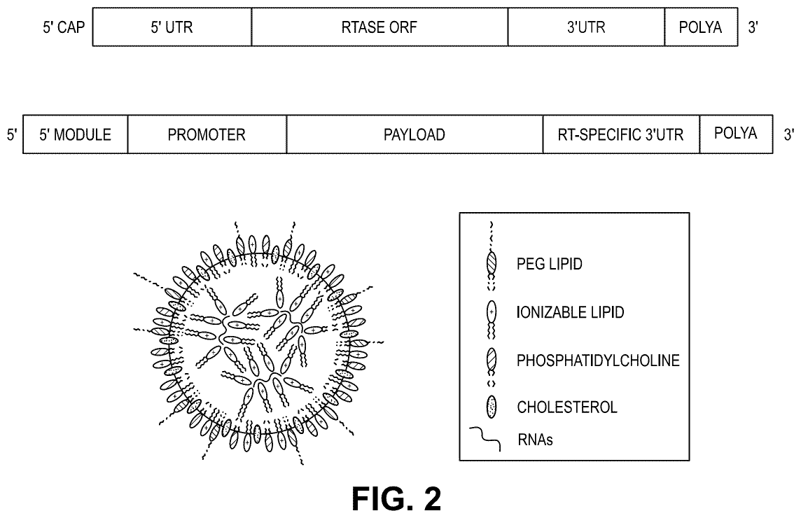

A diagram of an exemplary mRNA encoding an nrRT of the disclosure is shown in FIG. 2.

### Template RNA

In some aspects, the template RNA of the disclosure comprises (i) a promoter, (ii) a payload sequence, (iii) a polyA sequence, and (iv) a nrRT binding sequence. In some embodiments, the elements of the template RNA are operably linked to each other. It will be understood that the relative positions of the individual elements in the template RNA can vary in the 5′ to 3′ direction. For example, in some embodiments, the template RNA comprises, in a 5′ to 3′ direction, elements (i) (ii), (iii) and (iv). In some embodiments, the template RNA comprises, in a 5′ to 3′ direction, elements (iv), (i), (ii) and (iii).

It will be further understood that the individual elements in the template RNA can vary in their 5′ to 3′ orientation relative to other elements. For example, in some embodiments, the promoter (i) the payload sequence (ii) and/or the poly sequence (iii) are in a reversed 5′ to 3′ orientation relative to element (iv). Further, in some embodiments, the direction of transcription of the payload sequence in the template can be reversed, such that in one orientation the promoter (i) is closest to the 5′ end of the template RNA, or in a second orientation the promoter (i) is closest to the 3′ end of the template RNA.

A diagram of an exemplary template RNA of the disclosure is shown in FIG. 2.

In some embodiments, the promoter is an RNA polymerase (Pol) II promoter. In some embodiments, the promoter is selected from an EFS promoter, and ABPnat mini promoter, and CRNM-TTR enhancer promoter, an AAV-rDNA TTR promoter, or a CBh promoter.

In some embodiments, the payload sequence encodes a reporter protein such as GFP or luciferase. In some embodiments, the payload sequence encodes a therapeutic protein that replaces or complements a defective gene or protein. In some embodiments, the therapeutic protein is used to treat a disease or condition in a subject or patient. In some embodiments, the therapeutic protein is selected from the group consisting of Factor VIII, Factor IX, and phenylalanine hydroxylase (PAH).

In some embodiments, the payload sequence encodes a protein in the “gene name” column of Table 7 below. In some embodiments, the therapeutic protein is used to treat a disease or condition shown in Table 7 below.

In some embodiments, the payload sequence encodes an inhibitor of another protein. In some embodiments, the inhibitor is a single chain antibody.

In some embodiments, the payload sequence encodes a regulatory RNA. In some embodiments, the regulatory RNA is selected from a ligand-binding riboswitch, such as a ligand-activated riboswitch or an allosteric ribozyme (aptazyme), a small RNA (sRNA), a small interfering RNA (siRNA) or a short hairpin RNA (shRNA).

In some embodiments, the polyA sequence is selected from a short SV40 poly, SNRP1 polyA, a synthetic polyA, a BHG polyA, or a BGH polyA min. In some embodiments, the template RNA includes a WPRE3 3′ enhancer.

In some embodiments, the nrRT binding sequence comprises a sequence isolated from the 3′ region of a natural non-LTR retroelement or an organism comprising a non-LTR retroelement. In some embodiments, the nrRT binding sequence comprises a 3′UTR sequence. In some embodiments, the 3′UTR sequence is isolated from an organism comprising a non-LTR retroelement. In some embodiments, the 3′UTR sequence is isolated from an organism selected from the group consisting of G. aculeatus, D. melanogaster, L. polyphemus, P. pungitis, N. vitripennis, G. fortis, O. latipes, Z. albicollis, T. guttata, T. castaneum, T. guttatus, D. simulans, B. mori, and A. vaga. In some embodiments, the nrRT binding sequence comprises a sequence having greater than or equal to 60% sequence identity (e.g., greater than or equal to 60%, 65%, 70%, 75%, 80%, 85%, 90%, 95% or 99% identity) to a sequence isolated from G. aculeatus, D. melanogaster, L. polyphemus, P. pungitis, N. vitripennis, G. fortis, O. latipes, Z. albicollis, T. guttata, T. castaneum, T. guttatus, D. simulans, B. mori, or A. vaga. In some embodiments, the 3′UTR comprises a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 26-39.

In some embodiments, the nrRT binding sequence comprises a modified (non-natural) sequence. For example, the nrRT binding sequence can be modified to increase or decrease binding to an nrRT protein of the disclosure.

### Modified Uridines

In some embodiments, the mRNA encoding the nrRT and/or the template RNA comprises one or more modified uridine (U) nucleosides. RNAs containing unmodified uridines can activate the innate immune response and are less stable in cells. Modified uridines can provide the following advantages: i) they reduce the innate immune response in a host organism when cells are transfected with the nrRT mRNA and template RNA of the disclosure, ii) increase RNA stability, and iii) increase the amount of protein produced when the RNAs are transcribed.

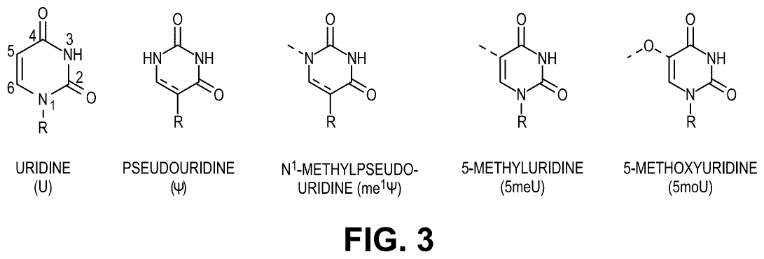

In some embodiments, the mRNA encoding the nrRT protein comprises one or more modified uridine (U) nucleosides, selected from the group consisting of N1-methyl-pseudouridine (N1mΨU), pseudouridine (ΨU), 5-methyluridine (5meU), 5-methyoxyuridine (5moU), and mixtures thereof. In some embodiments, the ORF encoding the nrRT comprises a modified uridine (U), selected from one of the following: N1-methyl-pseudouridine (N1mΨU), pseudouridine (ΨU), 5-methyluridine (5meU), or 5-methyoxyuridine (5moU). In some embodiments, the ORF encoding the nrRT comprises N1-methyl-pseudouridine (N1mΨU). In some embodiments, the ORF encoding the nrRT comprises a mixture or combination of unmodified uridines and modified uridines selected from the group consisting of N1mΨU, ΨU, 5meU, and 5moU. The structures of the modified uridines are shown in FIG. 3.

In some embodiments, the template RNA comprises one or more modified uridine nucleosides. The inventors unexpectedly determined that template RNAs comprising one or more modified uridines resulted in successful integration and expression of the payload sequence at a target site in the genome.

In some embodiments, the template RNA comprises one or more modified uridines selected from the group consisting of N1-methyl-pseudouridine (N1mΨU), pseudouridine (ΨU), 5-methyluridine (5meU), 5-methyoxyuridine (5moU), and mixtures thereof. In some embodiments, the template RNA comprises a single type of modified uridine selected from one of the following: N1-methyl-pseudouridine (N1mΨU), pseudouridine (ΨU), 5-methyluridine (5meU), or 5-methyoxyuridine (5moU). In some embodiments, the template RNA comprises N1-methyl-pseudouridine (N1mΨU). In some embodiments, the template RNA comprises a mixture or combination of unmodified uridines and modified uridines selected from the group consisting of N1mΨU, ΨU, 5meU, and 5moU.

In some embodiments, the template RNA comprising modified uridines is not cleavable by a ribozyme. In some embodiments, a template RNA comprising the modified uridines N1-methyl-pseudouridine (N1mΨU) or pseudouridine (ΨU) is not cleavable by a ribozyme. In some embodiments, a template RNA comprising a modified uridine increases the efficiency of insertion into the eukaryotic genome compared to template RNA comprising an unmodified uridine.

In some embodiments, cellular toxicity is decreased when the template RNA comprises a modified uridine.

It will be understood by a person of skill in the art that modified uridines are distributed throughout the template RNA sequence, and that in some embodiments, all the uridines comprise the same modified uridine (e.g., all the uridines are N1-methyl-pseudouridine (N1mΨU) or all the modified uridines are pseudouridine (ΨU)).

### 5′ Ribozymes

As is known in the art, native RNA templates that bind to their cognate nrRT protein comprise an active ribozyme at the 5′ end. The self-cleaving function of the ribozyme was previously thought to be critical for genomic insertion. Thus, in some embodiments, the template RNA further or optionally comprises an active or functional 5′ ribozyme sequence. In some embodiments, the 5′ ribozyme is selected from an HDV ribozyme (e.g., HDV_ac2, HDV_gu1, HDV_gu5b, HDV_gu6, HDV_gu5b_NP2), a TriCasA ribozyme, an L8 ribozyme (e.g., L8_gu6) an SL28 ribozyme, or a native cognate or semi-cognate ribozyme, or modified variants thereof. In some embodiments, the ribozyme sequence comprises a sequence selected from any one of SEQ ID NOs: 3 or 13 to 22 (without the pp7 binding sequence), or a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 3 or 13-22 (without the pp7 binding sequence).

However, in contrast to the teachings in the art, the inventors unexpectedly determined that template RNAs engineered to have 5′ ribozymes with reduced activity, catalytically inactive ribozymes, and ribozymes that are not cleaved could be successfully used to insert heterologous polynucleotides into a target site in the genomic DNA of a target cell. Thus, in some embodiments, the 5′ ribozyme sequence is selected from a partially active ribozyme, a ribozyme having reduced catalytic activity, or a catalytically inactive ribozyme sequence. In some embodiments, the template RNA does not comprise a functional 5′ ribozyme sequence. In some embodiments, the template RNA does not comprise a 5′ ribozyme sequence.

### Additional Components

In some embodiments, the template RNA, comprises, further comprises, or optionally comprises 5′ and 3′ elements that regulate transcription, translation, and/or insertion of the payload sequence at a target sited in the host cell genome. Non-limiting examples of these elements are described below.

In some embodiments, the template RNA comprises a Kozak consensus translation start site upstream or 5′ of the payload sequence. In some embodiments, the Kozak sequence comprises the sequence 5′-GCCACC-3′).

In some embodiments, the template RNA comprises an RNA polymerase (RNAP) terminator sequence located 5′ of the promoter sequence. The RNAP terminator sequence functions to stop RNA polymerase readthrough from genes at the target insertion site. In some embodiments, the RNAP terminator sequence comprises the sequence 5′-AGGTCGACCAGATGTCCGAGGTCGACCAGTTGTCCG-3′ (SEQ ID NO:4).

In some embodiments, the template RNA includes a 5′ sequence or 5′ modification that protects the 5′ end from degradation. In some embodiments, the 5′ modification includes a 5′ cap structure.

In some embodiments, the template RNA includes a 5′ sequence that promotes site-specific insertion of the heterologous polynucleotide into a target site in the eukaryotic genome.

In some embodiments, the template RNA comprises a 3′ sequence that promotes site-specific insertion of the heterologous polynucleotide into the eukaryotic genome. In some embodiments, the template RNA comprises a 3′ sequence that enhances the efficiency and fidelity of target-primed reverse transcription.

In some embodiments, the template RNA comprises a sequence useful for purification of the template RNA. In some embodiments, the sequence useful for purification of the template RNA comprises a hairpin structure that binds to the PP7 coat protein or a truncated version thereof. See, for example, Hogg, J. R. & Collins, K. RNA-based affinity purification reveals 7SK RNPs with distinct composition and regulation. RNA 13, 868-880 (2007).

In some embodiments, the template RNA comprises a sequence that binds to a DNA binding protein, which allows for enrichment of the inserted double strand sequences in the target DNA by purifying fragments of the genomic DNA comprising the sequence that bind the DNA binding protein. In some embodiments, the payload sequence is flanked by sequences that bind to a DNA binding protein, such that one sequence is located 5′ of the payload sequence (e.g., upstream of the promoter sequence), and another sequence is located 3′ of the payload sequence (e.g., downstream of the polyA sequence). In some embodiments, the template RNA comprises a lacO operator sequence that binds to the LacI protein. In some embodiments, the template RNA comprises a first lacO operator sequence located 5′ of the payload sequence and a second lacO operator sequence located 3′ of the payload sequence.

In some embodiments, the template RNA comprises a polyA sequence located 3′ of the nrRT binding sequence.

In some embodiments, the template RNA comprises a) a 5′ sequence that is homologous to a DNA sequence located 5′ to a target insertion site in the eukaryotic genome; or (b) a 3′ sequence that is homologous to a DNA sequence located 3′ to a target insertion site in the eukaryotic genome; or both (a) and (b). In some embodiments, the 5′ homologous sequence comprises about 1 to 36 nucleotides of homologous sequence that base pairs with a complementary sequence at the target site. In some embodiments, the 3′ homologous sequence comprises about 1 to 30 nucleotides of homologous sequence that base pairs with a complementary sequence at the target site.

In some embodiments, the template RNA does not comprise a 5′ phosphate.

### Methods for Inserting Polynucleotides at Target Sites in a Genome

The disclosure also provides methods for inserting a heterologous polynucleotide at a target site into a eukaryotic genome. In some embodiments, the method comprises transfecting a eukaryotic cell with: (a) an RNA encoding a non-LTR retrotransposon reverse transcriptase protein (nrRT) comprising a reverse transcriptase domain and an endonuclease domain; and (b) a template RNA. In some embodiments, the template RNA comprises, a promoter, a payload sequence, a polyA sequence, and a nrRT binding sequence.

In some embodiments, the template RNA comprises one or more modified uridine (U) nucleosides selected from the group consisting of N1-methyl-pseudouridine (N1mΨU), pseudouridine (ΨU), 5-methyluridine (5meU), 5-methyoxyuridine (5moU), and mixtures thereof. In some embodiments, the template RNA comprises mixtures of unmodified uridines, and one or more modified uridines selected from the group consisting of N1mΨU, ΨU, 5meU, and 5moU.

In some embodiments, the template RNA comprising modified uridines is not cleavable by a ribozyme.

In some embodiments, the nrRT is expressed in the cell and catalyzes insertion of a double stranded heterologous polynucleotide comprising the payload sequence at a target site in the eukaryotic genome.

The methods provide the advantage that template RNAs comprising modified uridines increase the insertion efficiency of the payload sequence into the eukaryotic genome compared to template RNA comprising unmodified uridines.

In some embodiments, the template RNA further comprises a 5′ ribozyme sequence selected from an active ribozyme. In some embodiments, the ribozyme is selected from an HDV ribozyme, a TriCasA ribozyme, a native cognate ribozyme, a semi-cognate ribozyme, or variants thereof.

The methods also provide the unexpected advantage that the template RNA does not require a functional ribozyme for insertion and expression of the payload sequence. Thus, in some embodiments, the template RNA comprises a 5′ ribozyme sequence selected from a partially active ribozyme, a ribozyme having reduced catalytic activity, or a catalytically inactive ribozyme. In some embodiments, the template RNA does not comprise a functional 5′ ribozyme sequence.

The methods also provide the unexpected advantage that the template RNA does not require a 5′ ribozyme sequence for insertion and expression of the payload sequence. Thus, in some embodiments, the template RNA does not comprise a 5′ ribozyme sequence.

Template RNA comprising modified uridines may also decrease cellular toxicity compared to template RNA comprising unmodified uridines. Thus, in some embodiments, cellular toxicity is decreased when the template RNA comprises a modified uridine selected from the group consisting of N1-methyl-pseudouridine (N1mΨPU), pseudouridine (ΨU), 5-methyluridine (5meU), 5-methyoxyuridine (5moU), and mixtures thereof.

In some embodiments of the method, increasing the molar ratio of the nrRT mRNA to the template RNA delivered to the target cell increases the insertion efficiency of the payload sequence at a target site in the genome compared to an equimolar (1:1) ratio. In some embodiments, increasing the amount of total RNA delivered to the target cell increases the insertion efficiency of the payload sequence at a target site in the genome. In some embodiments, increasing both the molar ratio of the nrRT mRNA to the template RNA and the total amount of RNA delivered to the target cell increases the insertion efficiency of the payload sequence at a target site in the genome. A representative, non-limiting example demonstrating the results of molar ratio of nrRT to Template RNA and total RNA on payload expression is described in the Examples.

In some embodiments of the method, the payload sequence encodes a therapeutic protein that replaces or complements a defective gene or protein. In some embodiments, the therapeutic protein is used to treat a disease or condition in a subject or patient. In some embodiments, the therapeutic protein is selected from the group consisting of Factor VIII, Factor IX, and phenylalanine hydroxylase (PAH).

In some embodiments of the method, the payload sequence encodes an inhibitor of another protein. In some embodiments, the inhibitor is a single chain antibody.

In some embodiments of the method, the payload sequence encodes a regulatory RNA. In some embodiments, the regulatory RNA is selected from a ligand-binding riboswitch, such as a ligand-activated riboswitch or an allosteric ribozyme (aptazyme), a small RNA (sRNA), a small interfering RNA (siRNA) or a short hairpin RNA (shRNA).

In some embodiments, the method comprises transfecting a eukaryotic cell. In some embodiments, the eukaryotic cell is transfected in vitro. In some embodiments, the eukaryotic cell is transfected in vivo. In some embodiments, the eukaryotic cell is a mammalian cell. In some embodiments, the eukaryotic cell is a human cell.

In some embodiments, the cell is transfected with a LNP formulation, a lipofection reagent, or by electroporation. In some embodiments, the cell is not transduced or transfected with a viral vector.

In some embodiments of the method, the template RNA, comprises, further comprises, or optionally comprises 5′ and 3′ elements that regulate transcription, translation, and/or insertion of the payload sequence at a target sited in the host cell genome. Non-limiting examples of these elements are described below.

In some embodiments, the template RNA comprises a Kozak consensus translation start site upstream or 5′ of the payload sequence.

In some embodiments, the template RNA comprises an RNA polymerase (RNAP) terminator sequence located 5′ of the promoter sequence. The RNAP terminator sequence functions to stop RNA polymerase readthrough from genes at the target insertion site.

In some embodiments, the template RNA includes a 5′ sequence or 5′ modification that protects the 5′ end from degradation. In some embodiments, the 5′ modification includes a 5′ cap structure.

In some embodiments, the template RNA includes a 5′ sequence that promotes site-specific insertion of the heterologous polynucleotide into a target site in the eukaryotic genome.

In some embodiments, the template RNA comprises a 3′ sequence that promotes site-specific insertion of the heterologous polynucleotide into the eukaryotic genome. In some embodiments, the template RNA comprises a 3′ sequence that enhances the efficiency and fidelity of target-primed reverse transcription.

In some embodiments, the template RNA comprises a sequence useful for purification of the template RNA. In some embodiments, the sequence useful for purification of the template RNA comprises a hairpin structure that binds to the PP7 coat protein or a truncated version thereof. See, for example, Hogg, J. R. & Collins, K. RNA-based affinity purification reveals 7SK RNPs with distinct composition and regulation. RNA 13, 868-880 (2007).

In some embodiments, the template RNA comprises a sequence that binds to a DNA binding protein, which allows for enrichment of the inserted double strand sequences in the target DNA by purifying fragments of the genomic DNA comprising the sequence that bind the DNA binding protein. In some embodiments, the payload sequence is flanked by sequences that bind to a DNA binding protein, such that one sequence is located 5′ of the payload sequence (e.g., upstream of the promoter sequence), and another sequence is located 3′ of the payload sequence (e.g., downstream of the polyA sequence). In some embodiments, the template RNA comprises a lacO operator sequence that binds to the LacI protein. In some embodiments, the template RNA comprises a first lacO operator sequence located 5′ of the payload sequence and a second lacO operator sequence located 3′ of the payload sequence.

In some embodiments, the template RNA further comprises a polyA sequence located 3′ of the nrRT binding sequence. In some embodiments, the template RNA does not comprise a 5′ phosphate.

In some embodiments, the template RNA comprises a) a 5′ sequence that is homologous to a DNA sequence located 5′ to a target insertion site in the eukaryotic genome; or (b) a 3′ sequence that is homologous to a DNA sequence located 3′ to a target insertion site in the eukaryotic genome; or both (a) and (b). In some embodiments, the 5′ homologous sequence comprises about 1 to 36 nucleotides of homologous sequence that base pairs with a complementary sequence at the target site. In some embodiments, the 3′ homologous sequence comprises about 1 to 30 nucleotides of homologous sequence that base pairs with a complementary sequence at the target site.

In some embodiments, the target insertion site is located in a ribosomal RNA gene or ribosomal DNA (rDNA). In some embodiments, the target insertion site is located in genomic DNA that encodes a ribosomal RNA (rRNA). In some embodiments, the target insertion site is located in a 5S, 8S, 18S, or 28S rDNA sequence.

In some embodiments of the method, the nrRT binding sequence comprises a sequence isolated from the 3′ region of a natural non-LTR retroelement or an organism comprising a non-LTR retroelement. In some embodiments, the nrRT binding sequence comprises a 3′UTR sequence. In some embodiments, the 3′UTR sequence is isolated from an organism comprising a non-LTR retroelement. In some embodiments, the 3′UTR sequence is isolated from an organism selected from the group consisting of G. aculeatus, D. melanogaster, L. polyphemus, P. pungitis, N. vitripennis, G. fortis, O. latipes, Z. albicollis, T. guttata, T. castaneum, T. guttatus, D. simulans, B. mori, and A. vaga. In some embodiments, the nrRT binding sequence comprises a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence isolated from G. aculeatus, D. melanogaster, L. polyphemus, P. pungitis, N. vitripennis, G. fortis, O. latipes, Z. albicollis, T. guttata, T. castaneum, T. guttatus, D. simulans, B. mori, or A. vaga. In some embodiments, the 3′UTR comprises a sequence having greater than or equal to 60% sequence identity (e.g., greater than or equal to 60%, 65%, 70%, 75%, 80%, 85%, 90%, 95%, 96%, 97%, 98%, 99% or 100% identity) to a sequence selected from any one of SEQ ID NOs: 26-39.

In some embodiments, the nrRT binding sequence comprises a modified (non-natural) sequence. For example, the nrRT binding sequence can be modified to increase or decrease binding to an nrRT protein of the disclosure.

In some embodiments of the method, the RNA encoding the nrRT comprises one or more modified uridine (U) nucleosides selected from the group consisting of N1-methyl-pseudouridine (N1mΨU), pseudouridine (ΨU), 5-methyluridine (5meU), 5-methyoxyuridine (5moU), and mixtures thereof, or comprises mixtures of unmodified U and modified U selected from the group consisting of N1mΨU, ΨU, 5meU, and 5moU.

### Safe Harbor Insertions Sites

In some embodiments, the heterologous polynucleotide is inserted at a so-called “safe harbor” site in the host cell genome, which does not alter normal cellular physiology or metabolism. Examples of safe harbor sites include regions of the genome with high copy numbers of repeated genes, such that disruption of one gene will not significantly alter normal cellular physiology or metabolism. Examples of high copy number regions include rDNA genes that encode rRNA. Thus, in some embodiments, the target insertion site is located in a ribosomal RNA gene or ribosomal DNA (rDNA). In some embodiments, the heterologous polynucleotide is inserted in genomic DNA that encodes a ribosomal RNA (rRNA). In some embodiments, the heterologous polynucleotide is inserted in a 5S, 8S, 18S, or 28S rDNA sequence.

### Delivery Methods

The compositions of the disclosure can be introduced into target cells using a method compatible with RNA delivery. In some embodiments, mRNA encoding the nrRT protein and the template RNA are introduced into the target cell using a lipid nanoformulation, such as a liposome or lipid nanoparticle (LNP), a lipofection reagent, or by electroporation. In some embodiments, the target cell is not transduced with a virus. Virus transduction is associated with various undesirable effects on cells, including mutations in the host cell chromosomes, random integration, and the presence of double strand breaks that can cause cellular toxicity.

### Pharmaceutical Compositions

Also provided are pharmaceutical compositions comprising the mRNA encoding the nrRT protein and the template RNA described herein. In some embodiments, the pharmaceutical composition comprises a lipid nanoformulation, such as a liposome or a lipid nanoparticle (LNP). In some embodiments, the pharmaceutical composition comprises a pharmaceutically acceptable excipient or salt. Examples of pharmaceutically acceptable excipients are described in the United States Pharmacopoeia (USP), the European Pharmacopoeia (EP), the British Pharmacopoeia, and the International Pharmacopoeia.

### Methods of Treatment

Also provided are methods of treating a subject or patient with the RNA compositions described herein. The methods can be used to treat a disease associated with a defective or mutated gene in a subject, such as but not limited to diseases caused by single-gene defects (monogenic disorders), such as Sickle cell anemia, Severe Combined Immunodeficiency (ADA-SCID/X-SCID), Cystic fibrosis, Hemophilia, Duchenne muscular dystrophy, Huntington's disease, Parkinson's, Hypercholesterolemia, Alpha-1 antitrypsin, Chronic granulomatous disease, Fanconi Anemia and Gaucher Disease. In some embodiments, the methods can be used to treat spinal muscular atrophy and inherited retinal dystrophy.

In some embodiments, the methods can be used to treat polygenic disorders, such as but not limited to Heart disease, Cancer, Diabetes, Schizophrenia, Parkinson's disease and Alzheimer's disease.

In some embodiments, the methods can be used to treat infectious diseases, such as HIV.

For example, in patients with hemophilia A, the payload can encode a wild-type factor VIII protein. In patients with hemophilia B, the payload can encode a wild-type factor IX protein. In some embodiments, the payload can encode a wild type p53 gene in a subject with a defective p53 gene to help prevent tumor growth.

Representative examples of diseases or conditions that can be treated by the methods of the disclosure are shown in Table 7.

In some aspects, the method is an in vivo method. In some embodiments, the method is an ex vivo method.

In some embodiments, the methods comprise administering an effective dose of a pharmaceutical composition of the disclosure to a patient in need of treatment. The pharmaceutical composition can be administered via any suitable method that results in targeted integration of the payload sequence into one or more cells of the subject. In some embodiments, the pharmaceutical composition is administered intravenously, intramuscularly, subcutaneously, intraocularly, intraretinally, within the CNS or other neural tissue, or intranasally.

Effective doses can range from 0.1 to 100 mg of active ingredient/kg body weight (including the end points and any subrange therein) of the subject or patient. An effective dose can also range from 1 microgram to 200 micrograms of active ingredient per dose (including the end points and any subrange therein) for an adult human. Effective doses can be readily determined by a skilled medical professional.

In some embodiments, the cell is removed from the subject or patient before being transfected ex vivo with mRNA encoding an nrRT protein and a template RNA of the disclosure. In some embodiments, the subject or patient is a human, a cell is removed from the human and transfected with mRNA encoding an nrRT protein and a template RNA of the disclosure. Following ex vivo transfection, correct insertion of the heterologous polynucleotide comprising the payload sequence can be determined, for example by amplifying sequences at the 5′ and/or 3′ insertion junctions, and/or amplifying the payload sequence. A correctly targeted insertion can also be determined by sequencing the genomic target site. Expression of the payload sequence can also be determined, for example, by detecting expression of a product encoded by the payload sequence, such as a protein or regulatory RNA. After correct integration and/or expression of the payload sequence is determined, the correctly targeted cells are administered to the subject (autologous therapy).

### EXAMPLES

The following examples are offered to illustrate, but not to limit the claimed invention.

### Example 1

This example provides a representative method for producing a template RNA comprising modified uridines.

Plasmid DNA used for in vitro transcription (IVT) to produce template RNA is digested using restriction enzymes BbsI-HF and PvuI to completion. The linearized plasmid is purified using phenol chloroform isoamyl alcohol (PCI) extraction and quantified using Nanodrop. The in vitro transcription is carried out using HiScribe T7 High Yield RNA Synthesis Kit (NEB, cat #E2040) in the presence of recommended quantity of T7 RNA polymerase mix, the corresponding reaction buffer, 50 ng/ul linearized plasmid DNA, 10 mM each ATP, GTP, CTP and the corresponding modified UTP. The reaction mixture is incubated at 37 C for 2 hours, followed by DNase treatment to remove DNA template. For each 20 ul IVT reaction, 2 ul of DNase I (NEB, cat #M0303S), 10 ul of 10× DNase buffer, and 68 ul nuclease-free water are added to 100 ul final volume. Incubate for 3 hours at 37° C.

Oligo(d)T25 magnetic beads (NEB Cat #S1419S) is used to purify the resulting RNA transcripts. For each 20 ul IVT reaction, 5 mg beads are used. The beads are equilibrated by washing three times with 250 ul of 1× Wash Buffer (20 mM Tris-HCL, pH7.5, 500 mM LiCl, and 1 mM EDTA). DNase-treated RNA is mixed with 2× Binding buffer (0.1% Triton X-100 in 2× Wash Buffer) at 1:1 (v:v) and mixed with the corresponding quantity of equilibrated beads by pipetting. Incubate at 37 C for 5 min and then incubate at room temperature on a rotator for 15 min. Wash beads three times with 250 ul of 1× Wash Buffer followed by washing one time with 250 ul of 1× Low-salt Wash Buffer (20 mM Tris-HCL, pH 7.5, 200 mM LiCl, and 1 mM EDTA). Elute RNA by adding 100 ul of nuclease-free H2O to the beads and incubate at 37° C. for 5 min.

CIAP (Promega Cat #M2825) is used to remove 5′ tri-phosphate from the RNA transcript. The eluted dT-purified RNA (100 ul) is mixed with 0.5 ul of CIAO, 30 ul of 5×CIAP Buffer, and 19.5 ul of nuclease-free water. The mixture (150 ul) is incubated at 37 C for 30 in and stopped by adding 6 ul 10% SDS and 1.5 ul 0.5M EDTA. Purify the treated RNA by PCI extraction and precipitation before resuspending into nuclease-free H2O at a volume that is equivalent to the original input volume of dT-purified RNA. The resuspended RNA is quantified using Nanodrop and checked for integrity by Tapestation.

### Example 2

This example provides a representative method for transfecting cells with an mRNA encoding a nrRT protein and a template RNA encoding the GFP reporter gene.

Prior to transfection, hTERT RPE-1 cells are lifted using Trypsin-EDTA (0.25%), phenol red (Gibco, 25200056) from a 30% to 50% confluent plate and seeded in a 6-well plate at a density of 500 thousand cells per well. Each transfection is done in duplicates. Dilute 10 uL of Messenger Max (Invitrogen Lipofectamine MessengerMAX, LMRNA003) in 250 μL of Opti-MEM and incubated for 10 minutes at room temperature. A total of 5 ug of a nrRT mRNA and a Template RNA at a molar ratio of 1:3 is diluted in 250 μL of Opti-MEM. The diluted RNA in Opti-MEM is then mixed with the diluted and incubated Messenger Max and incubated for 5 minutes at room temperature. The resulting mixture is then added into the two wells (250 uL each) seeded with the 500 thousand cells. Transfected cells are placed in an incubator at 37° C. with 5% CO2. Cells are imaged at Day 1 and Day 2 post transfection to assess cell health and transfection efficiency via image analysis. On Day 2, cells are washed with 1 mL of 1×PBS and 500 μL of Trypsin-EDTA (0.25%) and incubated for 3 minutes in an incubator at 37° C. with 5% CO2.

### Example 3

This example provides a representative method for analyzing ribozyme cleavage efficiency of Template RNA comprising uridine modifications.

A minimized version of the Template RNA (HDV_gu6_GFP) containing just the 5′ module sequence is produced following the protocol described in Example 1 with the uridine substituted at 100% by various modified uridines (see Table 4). After the completion of the DNase treatment step, 200 μl Oligo Binding Buffer is added to 100 μl post-DNase treatment RNA sample, which is then mixed with 800 μl ethanol (95-100%). Transfer ˜750 μL of the mixture to the Zymo-Spin IC Column (Zymo, Cat #D4060) positioned in a Collection Tube and centrifuge. Discard the flow-through. Transfer the remaining sample to the Zymo-Spin IC Column and centrifuge at 10,000-16,000×g. Discard the flow-through. Add 750 μl DNA Wash Buffer to the column and centrifuge for 1 minute ensure complete removal of the wash buffer. Carefully, transfer the column into a nuclease-free tube. Add 15 μl water directly to the column matrix and centrifuge. Quantify with Nanodrop. Run 25 ng purified RNA per lane on a 10% Criterion TBE-Urea Polyacrylamide Gel (Bio-Rad, Cat #3450089) at 120 V until bromophenol blue reaches the bottom of the gel. Add 1:10,000 dilution of SYBR Gold (ThermoFisher, Cat #S11494) in water to stain the gel by shaking at room temperature for 10 min while protected from light. Wash gel with water before taking images.

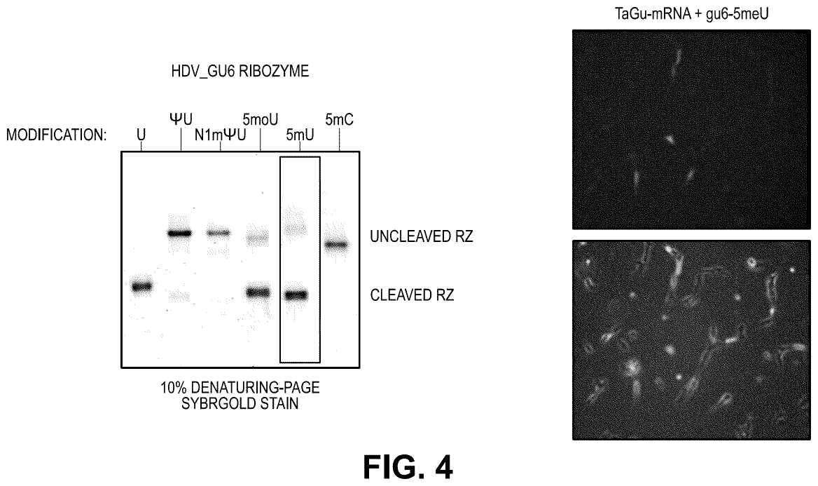

The cleavage efficiency is quantified using the densitometry analysis feature of ImageJ with background subtraction. The results (FIG. 4 and Table 2) show that the use of different uridine substitutions resulted in different efficiency of ribozyme cleavage. The use of unmodified uridine results in near-completion cleavage. The use of 5mU or 5moU leads to >80% ribozyme cleavage efficiency. In contrast, the use of 5mC, ΨU or N1mΨU resulted in very low to un-detectable cleaved product.

TABLE 2
Ribozyme cleavage efficiency with different uridine modifications.

| | U | ψU | N1mψU | 5moU | 5meU | 5meC |
|---|---|---|---|---|---|---|
| Cleaved | 8995 | 566 | 0 | 9780 | 10047 | 0 |
| Uncleaved | 195 | 9190 | 4450 | 2056 | 1128 | 6913 |
| Total | 9190 | 9756 | 4450 | 11836 | 11175 | 6913 |
| Cleavage | 98% | 6% | 0% | 83% | 90% | 0% |
| efficiency | | | | | | |

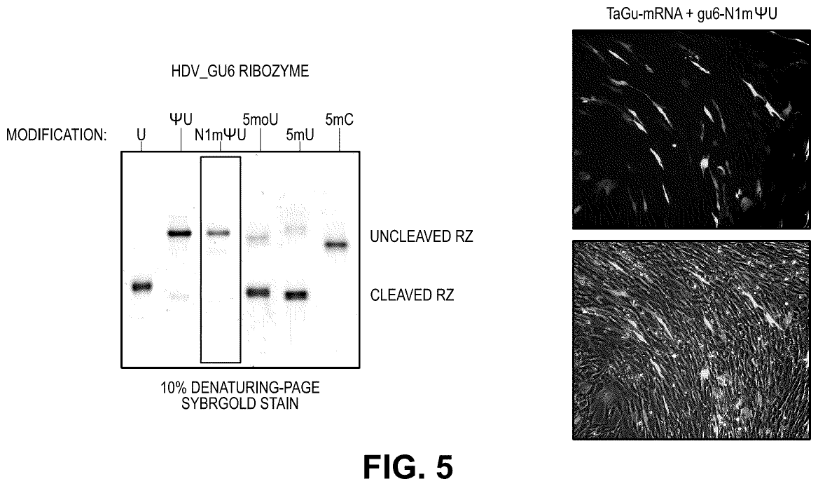

The corresponding full-length version of the HDV_gu6_GFP Template RNA containing either the 5meU or the N1mpU modification is co-transfected with the nrRT (TaGu RT mRNA) into hTERT RPE-1 cells using the protocol described in Example 2. GFP image analysis is conducted on Day 2 post transfection. The Template RNA with 5mU modification resulted in low payload integration as reflected by the low number of GFP positive cells as well as high cell toxicity (FIG. 4). In contrast, Template RNA with N1mpU modification resulted in significantly higher number of GFP positive cells and low toxicity (FIG. 5).

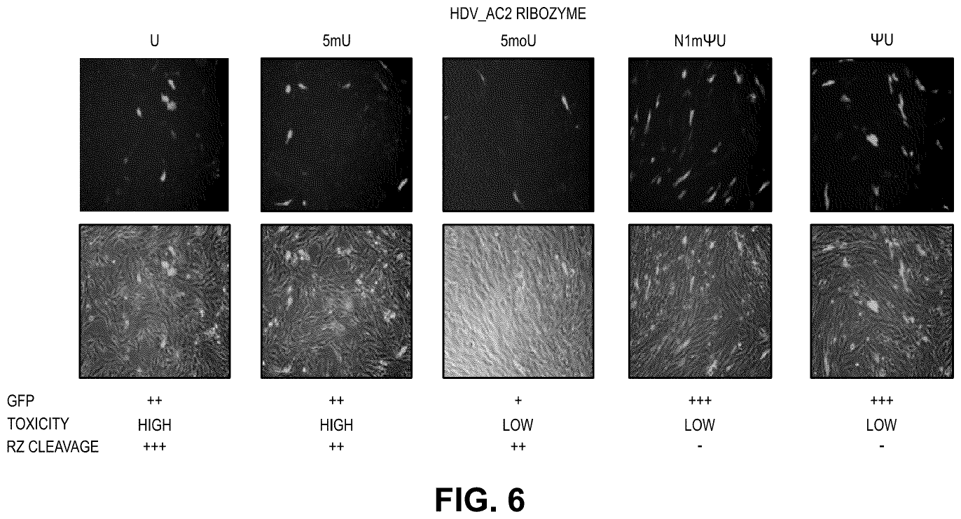

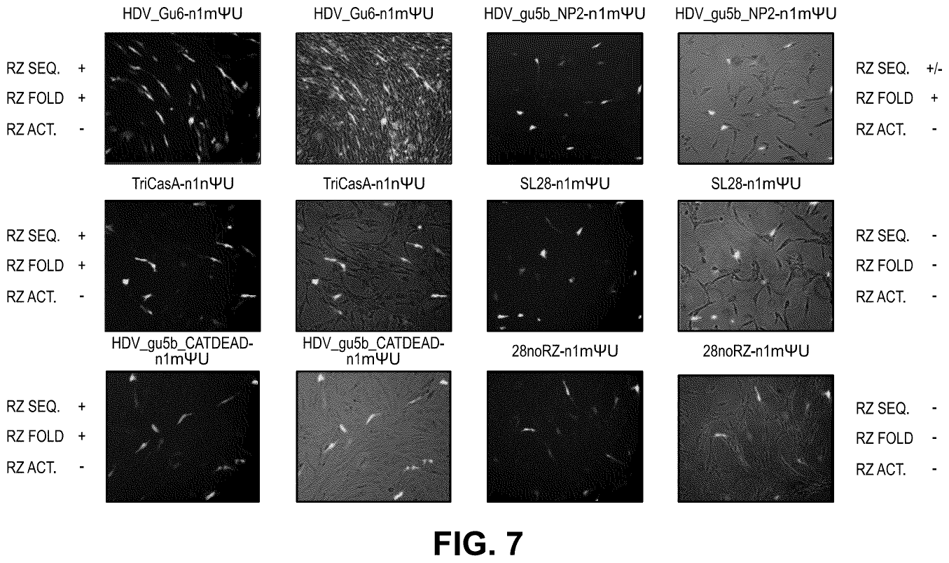

A second Template RNA, HDV_ac2_GFP, with different uridine modification, is produced using the protocol described in Example 1 and transfected into hTERT RPE-1 cells as described in Example 2 to assess the impact of uridine modification to the efficiency of payload expression and cell health. The results (FIG. 6) indicate that both unmodified U and 5mU lead to low number of GFP positive cells and high cell toxicity. The use of 5moU modification resulted in very low number of GFP positive cells and also low toxicity. Consistent with HDV_gu6_GFP Template RNA, the use of N1mΨU or ΨU resulted in significantly higher percentage of GFP positive cells without causing notable cell toxicity.

### Example 4

This example describes the junction analysis to assess the integration efficiency of the payload sequence at a target site in the genome and comparison the integration efficiency of Template RNA with uridine modifications.

gDNA Extraction and qPCR

Transfected cells are washed with PBS, pelleted by centrifugation, and flash frozen. Cells are lysed with Cell Lysis Buffer (0.1M EDTA, 0.5% SDS, 10 mM Tris-HCl pH 7.5, 0.2 mg/mL RNaseA) at 56 C for 10 minutes followed by 37° C. for 1-3 hours. An equal volume of phenol:chloroform:isoamyl alcohol (25:24:1) is added to cell lysate, vortexed at top speed for 10 seconds, and centrifuged at 21,000×g for 5 minutes at room temperature. The aqueous layer containing genomic DNA is removed and mixed with an equal volume of 100% isopropanol+300 mM sodium chloride and centrifuged at 21,000×g for 10 minutes to precipitate genomic DNA. The genomic DNA pellet is washed with 70% ethanol and centrifuged 5 minutes at 21,000×g. The genomic DNA pellet is air-dried for 5-10 minutes before resuspension in nuclease-free water. The total genomic DNA is quantified using the 1×DNA HS Quantification Assay Kit (Invitrogen Cat #Q33231) according to manufacturer instructions. Quantitative PCR is performed using NEB Luna Universal One-Step qPCR Kit (NEB Cat #M3003). Five nanograms of gDNA is used as template for each reaction, and each sample is run in technical duplicates or triplicates. Relevant forward and reverse primers are used at a concentration of 0.5 uM each per reaction. The cycling conditions are: 1 cycle of 95 C for 5 min, 40 cycles of (95° C. for 15 sec, 60° C. for 30 sec) followed by melting curve analysis step of heating from 65° C. to 95° C. Quantification analysis is done as described in the section below “qPCR Data Analysis.”

Cell-Direct qPCR

Transfected cells are washed with PBS and frozen at −80° C. in the tissue culture plate. Cells are lysed with Direct Cell Lysis Buffer (5 mM EDTA, 0.5% SDS, 10 mM Tris-HCl pH 7.5, 40 ug/mL Proteinase K) at 37° C. for 10 minutes. Cell lysate is diluted 1:1 with nuclease-free water, then heated at 37° C. for 5 min followed by 95° C. for 5 minutes. Cell lysate is further diluted 1:10 in nuclease-free water. Quantitative PCR is performed using NEB Luna Universal One-Step qPCR Kit (NEB Cat #M3003). Five microliters of diluted cell lysate are used as template for each reaction, and each sample is run in technical duplicates or triplicates. Relevant forward and reverse primers (see Table 3) are used at a concentration of 0.5 uM each per reaction. The cycling conditions are 1 cycle of 95 C for 5 min, 40 cycles of (95° C. for 15 sec, 60° C. for 30 sec) followed by melting curve analysis step of heating from 65° C. to 95° C. Quantification analysis is done as described in the section below “qPCR Data Analysis.”

qPCR Data Analysis

Quantification is done by setting a uniform fluorescence signal across all primer sets and samples and determining at what cycle number the fluorescence signal crosses the threshold for each well (referred to as the Cq value). The quantification of 3′ junctions from each sample is normalized to the quantification value of Tbp1 (a single copy gene) from the same sample by subtracting the average Cq value of Tbp1 from the average Cq value of the relevant junction to get a ΔCq value. The data are presented as a “fold over Tbp1” by transforming ΔCq: 2(−ΔCq).

TABLE 3
Primers used in junction analysis.

| Primer Name/Target | Sequence (5′ à 3′) |
|---|---|
| human Tbp1 Forward | CCGGAATCCCTATCTTTAGTCC |
| | (SEQ ID NO: 40) |
| human Tbp1 Reverse | TGCTGCCTTTGTTGCTCTTC |
| | (SEQ ID NO: 41) |
| 3′ junction Forward | AACGCGTCAATACCATCTGAC |
| | (SEQ ID NO: 42) |
| 3′ junction Reverse | GGAATCTCGTTCATCCATTCATG |
| | (SEQ ID NO: 43) |

Three different Template RNAs containing the five different uracil nucleotide (U, 5meU, 5moU, N1mpU, and pU) are transfected together with the TaGu RT mRNA into hTERT RPE-1 cells following the protocol described in Example 3. The cells are collected, and genomic DNA is extracted from each sample following the protocol described above. The qPCR-based junction analysis is done following the process described above. The results are summarized in Table 4. For each of the Template RNA, the use of N1mΨU or ΨU resulted in at least 2-5 fold higher 3′ insertion efficiency compared to that with the unmodified U. The other two modifications, 5meU or 5moU, resulted in similar 3′ insertion efficiency.

TABLE 4
Comparison of 3′ integration efficiency of Template RNA with
different U modifications.

| 5′ Module of | U | Ratio of 3′ junction to TBP |
|---|---|---|
| Template RNA | modification | (normalized to unmodified) |
| TriCasA-GFP | Unmodified | 1.0 |
| | 5meU | 2.0 |
| | 5moU | 1.0 |
| | N1mΨU | 4.0 |
| | ΨU | 5.0 |
| HDV_gul-GFP | Unmodified | 1.0 |
| | 5meU | 1.0 |
| | 5moU | 0.5 |
| | N1mΨU | 2.0 |
| | ΨU | 2.0 |
| HDV_ac2-GFP | Unmodified | 1.0 |
| | 5meU | 0.7 |
| | 5moU | 0.7 |
| | N1mΨU | 2.0 |
| | ΨU | 2.7 |
| HDV_gu5b_Catdead-GFP | Unmodified | 1.0 |
| | 5meU | 0.7 |
| | 5moU | 0.7 |
| | N1mΨU | 3.9 |
| | ΨU | 4.5 |
| 28NoRZ-GFP | Unmodified | 1.0 |
| | 5meU | 1.4 |
| | 5moU | 0.5 |
| | N1mΨU | 1.0 |
| | ΨU | 2.8 |

### Example 5

This example describes that a functional 5′ ribozyme in the template RNA is not required for integration of the payload sequence at a target site in the genome.

Template RNA containing a variety of 5′ module sequences (see Table 5) and encodes the GFP reporter gene as the payload are produced using the in vitro transcription (IVT) protocol described in Example 1. Uridines are substituted with N1mΨU in the IVT RNA. The resulting RNA is co-transfected with TaGu RT mRNA into hTERT RPE-1 cells as described in Example 2. The GFP image analysis of the transfected cells is summarized in Table 5. The results show that, with the N1mΨU modification, the integration of the GFP gene at the target site in the genome does not require the 5′ module of the Template RNA to contain an active ribozyme, a complete ribozyme structure, or any ribozyme sequence at all.

TABLE 5
Summary of the effect of 5′ module of Template RNA on gene insertion efficiency

| | | Contains | Contains | | | GFP+/ |
|---|---|---|---|---|---|---|
| | SEQ ID | Ribozyme | Ribozyme | Cleave | Cleave | insertions |
| 5' Module | NO: | Sequence | Structure | (U) | (N1mψU) | (N1mψU) |
| TriCasA | 13 | Yes | Yes | Yes | No | Yes |
| HDV_ac2 | 14 | Yes | Yes | Yes | No | Yes |
| HDV_gu1 | 15 | Yes | Yes | Yes | No | Yes |
| HDV_gu5b | 16 | Yes | Yes | Yes | No | Yes |
| HDV_L8_gu6 | 17 | Yes | Yes | Yes | No | Yes |
| HDV_gu6 | 18 | Yes | Yes | Yes | No | Yes |
| HDV_gu5b_catdead | 19 | Yes | Yes | No | No | Yes |
| HDV_gu5b_NP2 | 20 | Partial | No | No | No | Yes |
| SL28 | 21 | No | No | No | No | Yes |
| 28NoRZ | 22 | No | No | No | No | Yes |

### Example 6

This example describes that the molar ratio of the nrRT mRNA to the template RNA and/or the amount of total RNA delivered to the target cell influences the insertion efficiency.

Prior to transfection, hTERT RPE-1 cells were lifted using Trypsin-EDTA (0.25%), phenol red (Gibco, 25200056) from a 30% to 50% confluent plate and placed in an incubator at 37° C. with 500 CO2 until dilution series was done (no more than 30 minutes). Total amount of Messenger Max (Invitrogen Lipofectamine MessengerMAX, LMRNA003) was diluted into 140 uL (# of wells×30 uL×# of plates) of Opti-MVEM and incubated for 10 minutes. Total amount of Messenger Max needed was based on a volume to weight ratio of 2 uL Messenger Max to lug RNA. TaGu-RT mRNA and HIDV_gu5b-Luciferase-n1mpU RNA were mixed at specified molar ratios (see Table 6) and then diluted in 140 μL of Opti-MEM. The diluted RNA in Opti-MEM was mixed with the diluted Messenger Max and incubated for 5 minutes at room temperature. A serial dilution was done in a 96-well plate starting from the highest dose (1.25 ug) to the lowest (0.01 ug) per molar ratio across the rows of the plate. Twenty thousand cells were then added per well. A luciferase assay was performed using Bright-Glo Luciferase Assay System (Promega, Cat #E2620) on Day 1 and Day 2. For luminescence quantitation, Agilent's Cytation5 with Gen5 software was used with the following settings: Endpoint/Kinetic read type with a Luminescence fiber, gain at 135 with an integration time of 1 second and a read height of 4.50 mm. The on-platform mixing was achieved by clicking “Shake”, select “Linear” for shake mode with a duration of “0:04”. The intensity of the luminescent signal reflects the level of expression of the luciferase protein, which is the results of integration of the luciferase gene encoded by the Template RNA in the genomic site. The results are show in Table 6. The highest luminescence signal was observed when the molar ratio of nrRT to Template RNA was 1:6 and the dose of the total RNA was 0.08 μg/well.

TABLE 6
Effect of molar ratio of nrRT to Template RNA and total dose influence payload expression.

| Total RNA | Molar ratio of nrRT to Template RNA | | | | | | | | | | | |
|---|---|---|---|---|---|---|---|---|---|---|---|---|
| (ug/well) | 1:0.75 | 1:1.5 | 1:3 | 1:6 | 1:12 | 1:24 | 1:48 | 1:96 | 1:192 | 1:384 | 1:768 | 1:1536 |
| 1.28 | 5 | 6 | 6 | 19 | 9 | 5 | 6 | 5 | 11 | 10 | 8 | 3 |
| 0.64 | 10 | 7 | 20 | 9 | 47 | 45 | 11 | 5 | 5 | 47 | 23 | 6 |
| 0.32 | 112 | 90 | 65 | 178 | 96 | 69 | 87 | 7 | 10 | 139 | 117 | 113 |
| 0.16 | 502 | 315 | 327 | 432 | 591 | 360 | 284 | 298 | 355 | 299 | 185 | 143 |
| 0.08 | 766 | 711 | 862 | 1048 | 878 | 819 | 789 | 658 | 573 | 527 | 323 | 139 |
| 0.04 | 632 | 513 | 714 | 841 | 671 | 878 | 725 | 665 | 475 | 424 | 205 | 70 |
| 0.02 | 398 | 374 | 402 | 477 | 433 | 419 | 414 | 362 | 200 | 257 | 96 | 83 |
| 0.01 | 170 | 229 | 292 | 144 | 218 | 319 | 258 | 219 | 196 | 136 | 37 | 38 |

TABLE 7
Representative diseases and conditions that can be treated by the methods of the disclosure.

| Disease | Locus | Gene name |
|---|---|---|
| Achromatopsia (ACHM) | CNGB3 | beta 3 subunit of a cyclic nucleotide-gated |
| | | ion channel |
| Achromatopsia (ACHM) | CNGA3 | alpha 3 subunit of a cyclic nucleotide-gated |
| | | ion channel |
| Adrenoleukodystrophy | ABCD1 | ALDP protein |
| Albinism, oculocutaneous, type II | OCA2 | Oculocutaneous albinism II (OCA2) |
| Beta thalassemia | HBB | hemoglobin subunit beta |
| Brugada Syndrome | SCN5A | Sodium Voltage-Gated Channel Alpha |
| | | Subunit 5 |
| Canavan disease | ASPA | aspartoacylase |
| Charcot-Marie-Tooth Disease | PMP22 | Peripheral Myelin Protein 22 |
| Choroideremia (CHM) | REP1 | Rab escort protein 1 |
| Chronic granulomatous disease (CGD) | CYBA | p22-phox (phagocyte oxidase): alpha subunit |
| CILD1, with or without situs inversus | DNAI1 | Dynein, axonemal, intermediate chain 1 |
| (Kartagener syndrome) | | |
| Classical Ehlers Danlos (cEDS) | COL5A1/2 | Type V collagen |
| Cleidocranial Dysplasia (CCD) | RUNX2 | RUNX Family Transcription Factor 2 |
| Congenital deafness (presents at birth) | GJB2 | Gap Junction Protein Beta 2 |
| Crigler-Najjar syndrome, type I | UGTIA1 | bilirubin uridine diphosphate glucuronosyl |
| | | transferase |
| Cystic fibrosis | CFTR | CF transmembrane conductance regulator |
| Familial Adenomatous Polyposis | APC | APC Regulator Of WNT Signaling Pathway |
| Fanconi anemia | FANCE | FA Complementation Group E |
| Fragile X syndrome | FMR1 | fragile X messenger ribonucleoprotein 1 |
| Gaucher disease Type 1 | GBA | glucosylceramidase beta 1 |
| Hemochromatosis (iron overload) | HFE | Homeostatic Iron Regulator |
| Hemophilia A | F8 | Coagulation factor VIII |
| Huntington′s disease | HTT | Huntingtin (HTT) |
| Hypercholesterolemia, type B | APOB | apolipoprotein B |
| Hypophosphatemic rickets | PHEX | Phosphate-regulating endopeptidase |
| | | homologue, X-linked |
| Kneist Syndrome | COL2A1 | Alpha-1 chain of type II collagen |
| Leber congenital amaurosis (LCA) | CEP290 | centrosomal protein 290 kDa |
| Leber congenital amaurosis (LCA) | CRB1 | crumbs family member 1, photoreceptor |
| | | morphogenesis associated |
| Leber congenital amaurosis (LCA) | GUCY2D | guanylate cyclase 2D, membrane (retina- |
| | | specific) |
| Leber Hereditary Optic Neuropathy | ND4 | NADH dehydrogenase 4 |
| (LHON) | | |
| Leber Hereditary Optic Neuropathy | ND1 | NADH dehydrogenase 1 |
| (LHON) | | |
| Lesch-Nyhan syndrome (LNS) | HPRT1 | Hypoxanthine-guanine |
| | | phosphoribosyltransferase |
| Marfan syndrome | FBN1 | Fibrillin 1 |
| Medium-chain acyl-CoA dehydrogenase | ACADM | Medium-Chain Acyl-CoA Dehydrogenase |
| deficiency | | |
| Mucopolysaccharidoses (MPS) | IDUA | Alpha-L-Iduronidase |
| Muscular dystrophy, Becker type | DMD | Dystrophin |
| Muscular dystrophy, Duchenne type | DMD | Dystrophin |
| Myotonic dystrophy type 1 | DMPK | Dystrophia myotonica-protein kinase |
| Myotonic dystrophy type 2 | CNBP | CCHC-type zinc finger nucleic acid binding |
| | | protein |
| Neurofibromatosis types II | NF2 | Moesin-Ezrin-Radixin Like (MERLIN) |
| | | Tumor Suppressor |
| Neurofibromatosis, type 1 | NF1 | Neurofibromin 1 (NF1) |
| Niemann-Pick disease type A and B | SMPD1 | Sphingomyelinase |
| Parkison′s Disease | GBA | glucosylceramidase beta 1 |
| Phenylketonuria (PKU) | PAH | Phenylalanine hydroxylase (PAH) |
| Polycystic kidney disease 1 and 2 | PKD2 | Polycystic kidney disease 2 |
| Respiratory distress syndrome, Surfactant | SFTPC | Surfactant, pulmonary-associated protein C |
| protein-B (SP-B) deficiency | | |
| Retinitis pigmentosa visual field | EYS | Eyes Shut Homolog |
| Rett′s syndrome | MECP2 | Methyl-CpG-binding protein 2 |
| Rhodopsin-mediated autosomal dominant | PRPH2 | Peripherin 2 |
| retinitis pigmentosa (RHO-adRP) | | |
| Rhodopsin-mediated autosomal dominant | PRPF31 | Pre-MRNA Processing Factor 31 |
| retinitis pigmentosa (RHO-adRP) | | |
| Rhodopsin-mediated autosomal dominant | RHO | Rhodopsin |
| retinitis pigmentosa (RHO-adRP) | | |
| Sickle-cell anemia | HBB | hemoglobin subunit beta |
| Spermatogenic failure, nonobstructive | USP9Y | Ubiquitin-specific peptidase 9Y |
| Spinal muscular atrophy | SMN1 | Survival Of Motor Neuron 1, Telomeric |
| Stargardt disease | ABCA4 | ATP-binding cassette sub-family A |
| | | member 4 |
| Tay-Sachs disease | HEXA | Hexosaminidase A |
| Usher Syndrome | MYO7A | myosin VIIA |
| vitelliform macular dystrophy (Best) | BEST1 | bestrophin-1 |
| Von Hippel-Lindau (VHL) | VHL | von Hippel-Lindau ubiquitination complex |
| X-linked retinitis pigmentosa (XLRP) | RPGR | retinitis pigmentosa GTPase regulator |
| X-linked retinitis pigmentosa (XLRP) | RP2 | retinitis pigmentosa 2 |
| X-linked retinoschisis (XLRS) | RS1 | retinoschisin |
| α1-antitrypsin deficiency (COPD, | SERPI | α11-antitrypsin |
| emphysema,liver disease) | NA1 | |

It is understood that the examples and embodiments described herein are for illustrative purposes only and that various modifications or changes in light thereof will be suggested to persons skilled in the art and are to be included within the spirit and purview of this application and scope of the appended claims. All publications, sequence accession numbers, patents, and patent applications cited herein are hereby incorporated by reference in their entirety for all purposes.

### Informal Sequence Listing:

Template RNA sequences:
HDVRZ-28_gu5b_GFP_GeFo Full length Template RNA (SEQ ID NO: 1):
5′GGATAACCGCGTATGAGCGGTATCCTGGCGGGAGTAACTATGACTCTCTTAAGGA
AAAGAGAATCATAGAACGTCAGCAGCCTCCTCGCGGCCCCGCCGGTAACACAGAGG
AACACCCTGTGGCGAATGCTGACGATCTAGAAGGTCGACCAGATGTCCGAGGTCGA
CCAGTTGTCCGTGTGGAATTGTGAGCGCTCACAATTCCACACGTTACATAACTTACG
GTAAATGGCCCGCCTGGCTGACCGCCCAACGACCCCCGCCCATTGACGTCAATAGTA
ACGCCAATAGGGACTTTCCATTGACGTCAATGGGTGGAGTATTTACGGTAAACTGCC
CACTTGGCAGTACATCAAGTGTATCATATGCCAAGTACGCCCCCTATTGACGTCAAT
GACGGTAAATGGCCCGCCTGGCATTGTGCCCAGTACATGACCTTATGGGACTTTCCT
ACTTGGCAGTACATCTACGTATTAGTCATCGCTATTACCATGGTCGAGGTGAGCCCC
ACGTTCTGCTTCACTCTCCCCATCTCCCCCCCCTCCCCACCCCCAATTTTGTATTTA
TTTATTTTTTAATTATTTTGTGCAGCGATGGGGGCGGGGGGGGGGGGGGGGCGCGCG
CCAGGCGGGGCGGGGCGGGGCGAGGGGGGGGCGGGGCGAGGCGGAGAGGTGCGG
CGGCAGCCAATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGCTGCGACGCT
GCCTTCGCCCCGTGCCCCGCTCCGCCGCCGCCTCGCGCCGCCCGCCCCGGCTCTGAC
TGACCGCGTTACTCCCACAGGTGAGCGGGCGGGACGGCCCTTCTCCTCCGGGCTGTA
ATTAGCTGAGCAAGAGGTAAGGGTTTAAGGGATGGTTGGTTGGTGGGGTATTAATG
TTTAATTACCTGGAGCACCTGCCTGAAATCACTTTTTTTCAGGTTGGCGTACGGCCAC
CATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTAC
CCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCACAA
CGTCTATATCATGGCCGACAAGCAGAAGAACGGCATCAAGGTGAACTTCAAGATCC
GCCACAACATCGAGGACGGCAGCGTGCAGCTCGCCGACCACTACCAGCAGAACACC
CCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGT
GACCGCCGCCGGGATCACTCTCGGCATGGACGAGCTGTACAAGTAAAAGCTTGATC
CAGACATGATAAGATACATTGATGAGTTTGGACAAACCACAACTAGAATGCAGTGA
AAAAAATGCTTTATTTGTGAAATTTGTGATGCTATTGCTTTATTTGTAACCATTATAA
GCTGCAATAAACAAGTIGTGGAATTGTGAGCGCTCACAATTCCACAGCGGCCGCTG
AGGTAGATAATCTTTGTATAGTGGGGGGGGATCTCATGTACCGGGTTTCTTTTATTTG
ATTTTCAATAAAACAGACGGTAGCTAGGTTCGCAAGGCAGCCACAAGCCAAAGATA
GGTAGGGTGCTCATAGTGAGTAGGGACAGTGCCTTTTGATTCACAACGCGTCAATAC
CATCTGACACGGATACCCTTACCGGACTTGTCATGATCTCCCAGACTTGTCCAAGGT
GGACGGGCCACCTTTACTTAACCCGGAAAAGGAACATATATTAATTATATGTGTTCG
GAAAATAGCAAAAAAAAAAAAAAAAAAAAAA
pp7 sequence (SEQ ID NO: 2):
5′GGATAACCGCGTATGAGCGGTATCCT.
HDV_gu5b ribozyme with XbaI at the 3′ (SEQ ID NO: 3):
5′GGCGGGAGTAACTATGACTCTCTTAAGGAAAAGAGAATCATAGAACGTCAGCAGC
CTCCTCGCGGCCCCGCCGGTAACACAGAGGAACACCCTGTGGCGAATGCTGACGA(T
CTAGA)
Polymerase terminator. (SEQ ID NO: 4):
5′AGGTCGACCAGATGTCCGAGGTCGACCAGTTGTCCG
LacI binding site (SEQ ID NO: 5):
5′TGTGGAATTGTGAGCGCTCACAATTCCACA(LacO)
CBh promotor (SEQ ID NO: 6):
5′CGTTACATAACTTACGGTAAATGGCCCGCCTGGCTGACCGCCCAACGACCCCCGC
CCATTGACGTCAATAGTAACGCCAATAGGGACTTTCCATTGACGTCAATGGGTGGAG
TATTTACGGTAAACTGCCCACTTGGCAGTACATCAAGTGTATCATATGCCAAGTACG
CCCCCTATTGACGTCAATGACGGTAAATGGCCCGCCTGGCATTGTGCCCAGTACATG
ACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACC
CCCAATTTTGTATTTATTTATTTTTTAATTATTTTGTGCAGCGATGGGGGCGGGGGGG
GGGGGGGGGCGCGCGCCAGGCGGGGCGGGGCGGGGCGAGGGGCGGGGCGGGGCG
AGGCGGAGAGGTGCGGCGGCAGCCAATCAGAGCGGCGCGCTCCGAAAGTTTCCTTT
TATGGCGAGGCGGCGGCGGCGGCGGCCCTATAAAAAGCGAAGCGCGCGGCGGGCG
GGAGTCGCTGCGACGCTGCCTTCGCCCCGTGCCCCGCTCCGCCGCCGCCTCGCGCCG
CCCGCCCCGGCTCTGACTGACCGCGTTACTCCCACAGGTGAGCGGGCGGGACGGCC
CTTCTCCTCCGGGCTGTAATTAGCTGAGCAAGAGGTAAGGGTTTAAGGGATGGTTGG
TTGGTGGGGTATTAATGTTTAATTACCTGGAGCACCTGCCTGAAATCACTTTTTTTCA
GGTTGGCGTACG
Kozak sequence [symbol] : GCCACC
eGFP ORF (SEQ ID NO: 8):
5′ATGGTGAGCAAGGGCGAGGAGCTGTTCACCGGGGTGGTGCCCATCCTGGTCGAGC
TGGACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTAC
CCCGACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCA
AGGAGGACGGCAACATCCTGGGGCACAAGCTGGAGTACAACTACAACAGCCACAA
CGTCTATATCATGGCCGACAAGCAGAAGAACGGCATCAAGGTGAACTTCAAGATCC
GCCACAACATCGAGGACGGCAGCGTGCAGCTCGCCGACCACTACCAGCAGAACACC
CCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGT
GACCGCCGCCGGGATCACTCTCGGCATGGACGAGCTGTACAAGTAA
SV40 polyA with HindIII site at the 5′ (SEQ ID NO: 9:)
5′(AAGCTT)GATCCAGACATGATAAGATACATTGATGAGTTTGGACAAACCACAACT
AGAATGCAGTGAAAAAAATGCTTTATTTGTGAAATTTGTGATGCTATTGCTTTATTTG
TAACCATTATAAGCTGCAATAAACAAGTIGTGGAATTGTGAGCGCTCACAATTCCAC
A
GeFo 3′ UTR with NotI site at the 5′ (SEQ ID NO: 10):
5′GCGGCCGCTGA)GGTAGATAATCTTTGTATAGTGGGGGGGGATCTCATGTACCGGG
TTTCTTTTATTTGATTTTCAATAAAACAGACGGTAGCTAGGTTCGCAAGGCAGCCAC
AAGCCAAAGATAGGTAGGGTGCTCATAGTGAGTAGGGACAGTGCCTTTTGATTCAC
AACGCGTCAATACCATCTGACACGGATACCCTTACCGGACTTGTCATGATCTCCCAG
ACTTGTCCAAGGTGGACGGGCCACCTTTACTTAACCCGGAAAAGGAACATATATTAA
TTATATGTGTTCGGAAAA
r4 and polyA with BbsI site at the 3′ (SEQ ID NO: 11):
5′TAGCAAAAAAAAAAAAAAAAAAAAAAAA(GTCTTC)
G1. Luciferase ORF (SEQ ID NO: 12):
5′ATGGAGGACGCCAAGAACATCAAGAAGGGCCCCGCCCCCTTCTACCCCCTGGAGG
ACGGCACCGCCGGCGAGCAGCTGCACAAGGCCATGAAGCGGTACGCCCTGGTGCCC
GGCACCATCGCCTTCACCGACGCCCACATCGAGGTGGACATCACCTACGCCGAGTA
CTTCGAGATGAGCGTGCGGCTGGCCGAGGCCATGAAGCGGTACGGCCTGAACACCA
ACCACCGGATCGTGGTGTGCAGCGAGAACAGCCTGCAGTTCTTCATGCCCGTGCTGG
GCGCCCTGTTCATCGGCGTGGCCGTGGCCCCCGCCAACGACATCTACAACGAGCGG
GAGCTGCTGAACAGCATGGGCATCAGCCAGCCCACCGTGGTGTTCGTGAGCAAGAA
GGGCCTGCAGAAGATCCTGAACGTGCAGAAGAAGCTGCCCATCATCCAGAAGATCA
TCATCATGGACAGCAAGACCGACTACCAGGGCTTCCAGAGCATGTACACCTTCGTG
ACCAGCCACCTGCCCCCCGGCTTCAACGAGTACGACTTCGTGCCCGAGAGCTTCGAC
CGGGACAAGACCATCGCCCTGATCATGAACAGCAGCGGCAGCACCGGCCTGCCCAA
GGGCGTGGCCCTGCCCCACCGGACCGCCTGCGTGCGGTTCAGCCACGCCCGGGACC
CCATCTTCGGCAACCAGATCATCCCCGACACCGCCATCCTGAGCGTGGTGCCCTTCC
ACCACGGCTTCGGCATGTTCACCACCCTGGGCTACCTGATCTGCGGCTTCCGGGTGG
TGCTGATGTACCGGTTCGAGGAGGAGCTGTTCCTGCGGAGCCTGCAGGACTACAAG
ATCCAGAGCGCCCTGCTGGTGCCCACCCTGTTCAGCTTCTTCGCCAAGAGCACCCTG
ATCGACAAGTACGACCTGAGCAACCTGCACGAGATCGCCAGCGGCGGCGCCCCCCT
GAGCAAGGAGGTGGGCGAGGCCGTGGCCAAGCGGTTCCACCTGCCCGGCATCCGGC
AGGGCTACGGCCTGACCGAGACCACCAGCGCCATCCTGATCACCCCCGAGGGCGAC
GACAAGCCCGGCGCCGTGGGCAAGGTGGTGCCCTTCTTCGAGGCCAAGGTGGTGGA
CCTGGACACCGGCAAGACCCTGGGCGTGAACCAGCGGGGCGAGCTGTGCGTGCGGG
GCCCCATGATCATGAGCGGCTACGTGAACAACCCCGAGGCCACCAACGCCCTGATC
GACAAGGACGGCTGGCTGCACAGCGGCGACATCGCCTACTGGGACGAGGACGAGC
ACTTCTTCATCGTGGACCGGCTGAAGAGCCTGATCAAGTACAAGGGCTACCAGGTG
GCCCCCGCCGAGCTGGAGAGCATCCTGCTGCAGCACCCCAACATCTTCGACGCCGG
CGTGGCCGGCCTGCCCGACGACGACGCCGGCGAGCTGCCCGCCGCCGTGGTGGTGC
TGGAGCACGGCAAGACCATGACCGAGAAGGAGATCGTGGACTACGTGGCCAGCCA
GGTGACCACCGCCAAGAAGCTGCGGGGCGGCGTGGTGTTCGTGGACGAGGTGCCCA
AGGGCCTGACCGGCAAGCTGGACGCCCGGAAGATCCGGGAGATCCTGATCAAGGCC
AAGAAGGGCGGCAAGATCGCCGTGTGA
5′ Modules:
TriCasA (Bold sequences are pp7 sequence) (SEQ ID NO: 13):
5′GGAGACGGTCAACCGCGTAGGAGCGGTGACCGGAATTCGGCGGGAGTAACTAT
GACTCTCTTAAGGAGTCATAGAGCCAGAACCTCCTCGTGGTCCCGCTGGGCACAG
GGATTAATTTTTCTGTGGCAAATTTGACTGGCTTCAGAGAGCGTTTTTCGAAGTG
GACTGTGTGACTGCGTTCCCCCCTTAGTTGCTATATCCGCTTCGATTAACATCTC
ACCTCGACGTATAAGATCATT
HDV_ac2 (SEQ ID NO: 14):
5′GGAGAACCGCGTAGGAGCGGTCTCCTGGCGGGAGTAACTATGACTCTCTTAAA
AAAGAGAATCATAGAACGTCAGCAGCCCCCTCACGGCCCCGCCGGTAACACAGAG
GAACACCCTGTGGCGAATGCTGACGA
HDV_gu1 (SEQ ID NO: 15):
5′GGAGAACCGCGTAGGAGCGGTCTCCTGGCGGGAGTAACTATGACTCTCTTAAA
AAAGAGAATCATAGAACGTCAGCAGCCTCCTCGCGGCCCCGCCGGTAAGATTCCG
AAAGGAATCGCGAATGCTGACGA
HDV_gu5b (SEQ ID NO: 16):
5′GGAGAACCGCGTAGGAGCGGTCTCCTGGCGGGAGTAACTATGACTCTCTTAAG
GAAAAGAGAATCATAGAACGTCAGCAGCCTCCTCGCGGCCCCGCCGGTAACACAG
AGGAACACCCTGTGGCGAATGCTGACGA
HDV_L8_gu6 (SEQ ID NO: 17):
5′GGTAAACGGCGGGAGTAACTATGACTCTCTTAAAAAAGAGAATCATAGAACGT
CAGCGGCCTCCACGCGGCCCCGCCGGAACGCAGAGGAACACCCTGCGGCGAACGC
TGACGC
HDV_gu6 (SEQ ID NO: 18):
5′GGATAACCGCGTATGAGCGGTATCCTGGCGGGAGTAACTATGACTCTCTTAAA
AAAGAGAATCATAGAACGTCAGCGGCCTCCACGCGGCCCCGCCGGAACGCAGAGG
AACACCCTGCGGCGAACGCTGACGA
HDV_gu5b_CatDead (SEQ ID NO: 19):
5′GGCGGGAGTAACTATGACTCTCTTAAGGAAAAGAGAATCATAGAACGTCAGCA
GCCTCCTCGCGGCCCCGCCGGTAACACAGAGGAACACCCTGTGGAGAATGCTGAC
GA
HDV_gu5b_NP2 (SEQ ID NO: 20):
5′GGAAAAGAGAATCATAGAACGTCAGCAGCCTCCTCGCGGCCCCGCCGGTAACA
CAGAGGAACACCCTGTGGCGAA
SL28 (SEQ ID NO: 21):
5′ GGCGGGAGTAACTATGACTCTCTTAACAAAGAGAGAATAGTAACTCCCG
28NoRZ (SEQ ID NO: 22):
5′GGCGGGAGTAACTATGACTCTCTTAA
TaGu native ribozyme (T. guttata) (SEQ ID NO: 23):
5′GGCGGGAGTAACTATGACTCTCTTAAGGGTCTAGTTACAACTGGGCATCGCTGCA
GAGATCGCACCTCCTCGTGGTCCCGCTGGTAGCCCTTCGAAGGGTGACTAAGTCGAT
CTCTGCCCCAGGTACGGAGCCGTTGGGACTCACCAGTCCAACGTAACTCCTGCCTAA
ATTCGGTGAAACAAATTCCTCGGTAAAAAGCCCCATGGCTTCTTGCCCGAAACCTGG
CCCCCCGGTTTCAGCAGGGGCAATGAGTTTGGAAAGTGGACTGACCACCCACTCCGT
TCTCGCCATCGAACGTGGTCCCAATTCGTTGGCAAATTCCGGATCAGACTTTGGGGG
GGGGGGTCTGGGGCTACCGTTACGCCTATTGAGGGTATCGGTCGGCACTCAGACCTC
CCGCTCCGACTGGGTAGACCTGGTGTCCTGGAGCCACCCAGGACCCACGTCTAAGTC
CCAGCAGGTTGACCTGGTGTCTTTATTTCCTAAACACCGGGTTGACCTGTTATCCAA
AAACGACCAGGTAGACCTGGTGGCTCAATTTTTACCATCTAAATTTCCCCCCAATTT
GGCAGAAAATGATTTGGCTTTGCTGGTGAACTTAGAGTTCTACAGATCGGATTTGCA
TGTGTATGAGTGTGTTCATTTTGCTGCACATTGGGAGGGATTAAGTGGTTTGCCTGA
GGTGTATGAACAACTTGCACCACAACCGTGTGTGGGAGAAACTTTACATTCTAGCCT
CCCACGAGACAGTGAACTGTTTGTGCCTGAAGAGGGGAGCAGCGAGAAGGAGAGC
GAGGACGCGCCAAAAACATCTCCTCCGACGCCTGGGAAACATGGTTTGGAACAGAC
TGGGGAGGAAAAAGTG
TiGu native ribozyme (T. guttatus) (SEQ ID NO: 24):
5′GGCGGGAGTAACTATGACTCTCTTAACTGGGGACCGTGGTTACAACCCGGGCTTA
GCTGCAGAGACAGTACCTCCCCGTGGTTCCCGCCGGACCCCGTAACATCGGGTGACT
GAATCTGTCTCTGCCCCGGGAGTAGTTCCTCCTTGCCCTATTGACCAGCGGTCGCCG
GCTGCTCAATAGTATTCTAGGCGTGAAATATAGCGATAGTCCTAGTGGTTGTCTTAC
TGGGCCATAGCCCCTTGCTTCAGGGGTCATTCGCGAAGTCTCTCAGGAGAACTGGGG
GTGGTGTTCTTCTGGGTATAGCTAAACCCCCTAGACTGTGTCCGATCCATGGGGTCC
TGGATCGTGAATTTCGTTTCGGTGGCGACTCAGACGGGAGAATTCCCTGTGGATACG
GCCAGGAGGGCACCTGTGCCGGTAACATCATACCCTGAGTCGGAATGCCACATACC
GTTGCCCCTGACATTTTGTAACTCGGATGTGACTATTTGGGGAGGGGTTCGCCCTGA
ACCGGTGGACTGCTTGGGTGATCTTCCAGAGGTGTATGATGCACTCCCAGGGGTGGC
TGGGCCTCGGGAATCGGTGGGTGGGAGCCCGCCGGGAGAAGGGGTCAGGTCGCCAG
GGATTGCGTCACCCTCTGGTACTGCGGTCCAACATGATTTTGGGAGTCCCATCCTCG
TACCGG
ZoA1 native ribozyme (Z. albiollis) (SEQ ID NO: 25):
5′GGCGGGAGTAACTATGACTCTCTTAAGGCGACTTGAGAAGGTCTGGTTACAACTG
GGCATAGCTGCAGAGATCGCGCCTCCTCGTGGCCCCGCTGGTAAGCCCTTAACAGG
GTGACTAAGTCGATCTCTGCCCCAGTCCAGGAGCCGCTGGGTTTCACCAGCCCAGCG
ATTCCTTCCAAATTCGGTGAAACAAATTCCTCGGTAAAAGCCGCGTGGCTTATTGCC
TGAAACCTGGCCCCCCGGTTTCAGACAGGGGCAAAGAGTTCGGAAGTGGACTGACC
ACCCACCCCGAACCCGAGAGCGAATCTGGTCATGACCCAACTGTCCCAAATCCTGGT
CCGTCTCTTGGAGCGGGGGAAGGTGCACAGCCACTACCCTTACTCAGGGTATCGGTG
GGCACCCAAACCTGTGAAGAGGACTTTATAACATCTAGACCAACCAAATTACCCGG
AATTGAATCAGAATTAGGCCCGCTGGTGAAGTTTTCTTTAGAGGTTTACAGGTCAGA
TCTTAAGGGGGATGTGCAATTTGAGGGGATTCATTTTCCAGATAATTGGGGGGTACT
GGAGGGGTTTCCTGAGGTGTACGAACAACTGGCACCACAGCCAAACGGGGGAGACG
AGTTAAATCATAGTCTCCCAGGGGACAGGGAGGGGGATGTACTTGAGAAGGATAGC
AGCGAAAAGGAGAAGGAGGCTGCACCAGAGGCATTGCCCTCAGTGCAAAGGGCCC
GCAGTGAACAGTTGCC
III.
3′ Modules:
GeFo 3′UTR (G. fortis) (SEQ ID NO: 26):
5′GGTAGATAATCTTTGTATAGTGGGGGGGGATCTCATGTACCGGGTTTCTTTTATT
TGATTTTCAATAAAACAGACGGTAGCTAGGTTCGCAAGGCAGCCACAAGCCAAAGAT
AGGTAGGGTGCTCATAGTGAGTAGGGACAGTGCCTTTTGATTCACAACGCGTCAATA
CCATCTGACACGGATACCCTTACCGGACTTGTCATGATCTCCCAGACTTGTCCAAGG
TGGACGGGCCACCTTTACTTAACCCGGAAAAGGAACATATATTAATTATATGTGTTC
GGAAAA
ZoA1 3′UTR (Z. albiollis) (SEQ ID NO: 27):
5′TAGGTAGTCACATTGCACTTTCTGTAACTTGCACTGGGTGTGGGATGTGGGCCTG
GGGTGTGGGTTATGGGGTATATATGTGGGATATTCTGGTGGGAATGTCCATTCACTG
TATGCCTATCTTTTTAATAAAAAGACGGTAGCTAGGTTCGCGAAGCAGCCACAAGCC
AATAGCCAGTTAGGTAGCTCATAGTGGGTAGGTGACAGGAACCTTTGACTCAGAACG
CGTCCATTAACATCTAGAACGGACCAAACTTCGGACATGCACCGATTAACCGGATTT
GTCCAAGGTGGACGGGCCACCTTTACTTAACCCGGAAAGGGAACATATATAGTTAT
ATGTGTTCGTAATA
TaGu 3′UTR (T. guttata) (SEQ ID NO: 28):
5′TAATTCAGGTTATTTAGATGCTTAGTTTTTGTACCTTTCTTGTTTTGTTTAGGAT
TTTGATAGTGTTAGTATTTTTATATTTTTGTACGATTGCATAATGTTCTTTTTTATA
CAGTTCTGTTTTAATAAAATAGACGATAGCTAGAGACGTTAGGGCAGCCACAAGCCA
GTTAGGTAGCGGATAGTAGGTAGGAACAGACTTTTACTATTTCATAACGCGTCAATT
ACCACCTGATTTGGACCAATTCACGGGATTTGTCCAAGGTGGACGGGCCACCTTTAC
TTAACCCGGAAAAGGAACATATATAATTTATGTGTGTTCGATAAA
TiGu_3′UTR (T. guttatus) (SEQ ID NO: 29):
5′TAGGGGGCTTGGCATTTCTCATTGCCTGCTCCTGAAAGGATATGGGTCCTGCGTCG
CGTGGTAGGCAGACCCATTCGTCCGAGTAGGGGGCTTGGCAGTNTCCATTGCCTGTG
CCCGAAAGGACGTGGGTCATCTGGTCTGTCTGCCTACACCTCTCTAGACTTGTAACA
TCTAGTCTGTCAACAAGATCAAAATTCTTCACACAGACGACCGAGCTTGCTCAGTCT
TCCTGTACCCGCAGAATTTTGCTCTTGCTCTCCTTTGGCTGTGTCCTGGACGTGGGAC
TATTCCATCTCGTCCCAAATGCCGCGTCCAATTATACCGGATTTGACAAAGCGGACG
GCCCGCTTTATAAGCCGGAAAAGGTGCCTTGTAAAATTGCAAGGTTCATTAAATAG
BoMo 3′UTR (B. mori) (SEQ ID NO: 30):
5′TGAGCCTTGCACAGTAGTCCAGCGGTAAGGGTGTAGATCAGGCCCGTCTGTTTCTC
CCCCGGAGCTCGCTCCCTTGGCTTCCCTTATATATTTTAACATCAGAAACAGACATTA
AACATCTACTGATCCAATTTCGCCGGCGTACGGCCACGATCGGGAGGGTGGGAATCT
CGGGGGTCTTCCGATCCTAATCCATGATGATTACGACCTGAGTCACTAAAGACGATG
GCATGATGATCCGGCGATGAAAA
OrLa 3′UTR (O. latipes) (SEQ ID NO: 31):
5′TGAGGGGGACAGCTGGGAGTCTCGGCATGATTACAAATCTTGCGCTGCACTCGGA
TGTCGTCCCCGTGACGGACACATTAATCCGGAAAGCGAGTGGTGACTCGCCTCAAG
TriCasB 3′UTR (T. castaneum) (SEQ ID NO: 32):
5′TAAAATCTCCTGACCAACTAGCTCACTGACTAATTTTAAACTGTCCTGTCTTACTTG
TTTTACACGTGCTCTGTGGCGGGGCCATTTACACCCCGTCGCAACACAACCTGTAAA
TACTTGTGTATGTCTGTTTATGTCCTAATTTATTATTTTAAACAGATCTTGGCCATGG
TCTCGGCCAACCAATTAAAGTCAGTGATGCGAGTCGCAATGCGGAGCAAGAGACCT
AGGCGTGTATTTATTGCTGGCATGCGGCGCCGGAGCCGGTCATCTGCTATGGGGAGC
AATGGCCGGGCGGATACCTCCACGTGGTTCCCTGTGGGTGGCCCGTCGAGGACGGT
AACCAGCGAAACTCCGTAAAGTCCTTCTTACGAGAAGGAACTCCGGTTAAAGATTTT
TCCAAGCCTGTACACGTGATTCCCTTGGAACAAGCAAAGTGTGGTTCCCTCGAGAGG
GCCCAGGTCAGGAGTTCGCAATAGTGGGCTGCAAGAGTTCATGCTGGGCTACAGTG
TCAGGACGAAGAGTGGGTAGTGATCGCAAAATCACGTGAATAGCTACCCCCCGCCT
GGCACCACTAGACAACAACAAGGGGTACGACAGCTCTTCTGTCGAAAGTTCGGGCG
CACACCCGTAAAAGG
DroSi 3′UTR (D. simulans) (SEQ ID NO: 33):
5′TAGCTAAAACGTTTGGTTCAAAACATTTGCTTGCTGTCTTGGCATAACATCAATAA
AGGCATAAACATCGCAAAATAATGGTTATATATAAATGGCTATGAGGATGGTTTTAG
TACGTAGGCGTTGCGGAACTTCGGTTCAGATAGAGCAATGAATCGTGCATGCTAGG
AAAACTGACCACACGCAGTGTTGGCAGCCCTAGTATCTTTCGATAGATTTCCATACC
TCCGCGATCAAAAAAAAAAAAAAAAAAAAAAA
Pupu 3′UTR (P. pungitis) (SEQ ID NO: 34):
5′TAGGGTTCCTCCACCCTCCGGCTGACGAACAGCTGGTAAACGGGGGGGCGGTGGG
GTGCCTCTCCAGCCGACTGATACAGGAGTAAGGGACGGTGGGGTCGCATCCAGGAA
GCGCAGCACCGCGATGCCGAAACTGATGTGCAGTATAACACAGAAAGCCTAAAGGG
CCAAAAG
Lipo 3′UTR (L. polyphemus) (SEQ ID NO: 35):
5′TAAATTTTGTCTCTTTCCCCAATGATGTCTACTAGCACGCTGCCGAAGCTAGATAG
ATTGAGGAATCTGCGTAATCTGTAATGATTACGCCTCATGGGCATCTATCGGTAGCG
TCGACCCTGACGTTAAATTGGGT AATAAGAAATAT
Navi 3′UTR (N. vitripennis) (SEQ ID NO: 36):
5′TGACCTGAACAAAACGTGTTGTCTTGTCTTGTCTAAAACTATTTATTCGAAATAAG
GGGAGGCTAACTGCCTGCAAGTTGAACGCGAAAGTTAGACCTTCCCACCTAAAGCC
CAAAAGTGATCGGGGAATGAATCCGCGGGTGACCCCAGAGTTGGGTAAACCCTTGA
AACGTTGGAGAAGCGGAAGAGAGTCCCGCCACCGAGCATCGAGTGCTGCGGCGCCC
GAATGAAACCGATCGCGGATGGTGCAAGTCGTAGGACGGGGCACGACCTAAGCCTC
TGTCACGGCGGCGAAGCCAGGAATCACCATGCAAAGGTGTGAACTGGGGCGGATAC
CTCCACGGGGTTTCCCTGGGCATCGCGCGAGCGATGGCCAAAGTCCGCTTTCTCAGC
TACAAAACAAAAATGGTATGAGACTTCGTTAACACTAATTTTTCCGAGCCTAGCAGG
CTCCCTTGACAACGCTTATGAATCTGGAAAAGGACACAAAGTGGAAAAAGCGCTGA
TGGTGGACAAAAGTCAGTTGAGACTTGATATCAGTTGTTTTGACTAAGAATTTTATT
ATCGTTGACTTTTAAATATTTTATTATTGACTGTTAATATACTGACTTGGGACCAAGT
CATCTCTGTTACCCGGTACCGGTTCCTGTCATCAAACCGGAAAGTCCGTCCCACGTA
ATGTGGTAGACGCAGGAG
GaAc 3′UTR (G. aculeatus) (SEQ ID NO: 37):
5′GGAGGGGAGTAGGTCTCTACTCTGACCCGAAGGGCCCCCCCGTTTCAGACCTGAT
TCTAGGCTACCTGTGCCTAATTGGGGGGGTCCCAAAGAGATGTTGTCTGTTGTAGAA
GGGTTTGCGCCACTGACTGCACGGAAGGGTGGGCCTCGACAGGTAGGGGTTACATG
ACTCCGTGCTGCTCAGCAGACCCGCGCCTCTGAGACCGGGTAGGGCTACTTGAACA
AGCGACGCCCTGGTGTATGTCCGTATCCTAACCTGGTTTGGGAAAGCCGATACCGGC
AATGCCCGCCACAGGTGTCGCGCACCCCACGGGATGACGTATGGGCCCCGGGGGAC
CTCATGGATACTCCACTGGACTTGCACAATCCTGGTGTACTGGATGCAGCGACGTTG
GTGACATAAGCAATCGCTAAGTCGGGGTAGGGGAGGTGGGGACCTCGGCACGGCTG
TAGGAACGGGTGTATGGGCTCCGGCAGCCGTCGTCACTCCCATACAACACAGGGGC
TGCATCCTGGTGGCCGGTGCTAGTTGGTTCTGGAAGCCCGCCCGGGCTGGTTCGCAG
AAGCAGGGTGCGCCCAGGGTAGGTTTGGTATATCTGGGTCCGGTGCGATACCTATCG
ATGGGCAGCGAGGGCCGCCTCGTGACGCGCTGTGTGGAGCTGGAGCCGGCCTGGGT
ATGAACAGTTCTTGCGGATGTGGCGTAGCTAGATAGTACCCGTGGTTGTGGGCGTGG
TGTCGACCAAATGTTGTCCTGTGTGCACATAGGCCAAGGGTTACGTGGGTGGCAGTC
AGAAGCACCCGCACCTGGAAGTGATTGCCCCGGGATCCCGGCTCTCTGTGAAGAGC
TACCTTGAGGAAAGGTGTTCCGCTGGAACTCAAGACCCTACAGTAGGGGATATCAA
CTGGCTTTGAGGTGCTGTGATTCCGGAACCAGGGCGAGGGCGAGTACTTAGAGCAT
GTCCAAAAGCCCGGGGAACGTTCCGGGGGCCTGCTTGGGTCGTTGGACCCACATCC
GTAAAACGATGGATCTCGCGTCGGCGCTCGGGAGAACTTCCCGCATGAACGCTGATT
GCATGTGAGAACGCCCCCACGGCGGCGGGGCAGGCGCTCCCCCTGGGTGTAAGGCT
CGGGGGGGTCACGGCTCCGCTCTAAAAG
DrMe3′UTR (D. melanogaster) (SEQ ID NO: 38):
5′TAGCTAAATCGTTTGGTTCAAAACATTTGCTTGCTGTCTTGGCATAACATCAATAA
AGGCATAAACATCGCAAAATAATGGTTATAATTAAATGGCTATGAGGATGGTTTTAG
TACGTAGGCGTTGCGGAACTTCGGTTCATATAGAGCAATGAATCGTGCATGCTAGGA
AAACTGACCACACACAGTGTTGGCAGACCTAGTATCTTTCGAAGATTTCCATACCTC
CGCGATCAAAAAAAAAAAAAAAAAAAAAA
AdVa3′UTR (D. MELANOGASTER) (SEQ ID NO: 39):
5′TGAACTAGTCTCCTTCTTCTATTAGTCAGTCTAATTAATTTTTCTTACATTCTACATC
TAGTTCCATTATTAAATTGGTATGATCAGTGCTATCTCTGCTACACTCAATGCTTAAT
CGTATGTTATTGACAGTCTGACACTTGATTACTCTTACGACATATGCACTGTTTGCTT
CAGAGAAACCACTGTTCATATAGTGAAGTTCCTCAGTTTTCTGTTGATATATTCTTCT
TTCATTCTCGCTTCTCCTTTTCTACTGTGTTCTTTTTATCAGTTTTTTGTGGAAAAATT
GAGAATAAATAAAGT

1-55. (canceled)

56. A composition comprising:
(a) an RNA encoding a non-LTR retrotransposon reverse transcriptase protein (nrRT) comprising a reverse transcriptase domain and an endonuclease domain; and
(b) a template RNA, wherein the template RNA comprises:
(i) one or more modified uridine (U) nucleosides selected from the group consisting of a N1-methyl-pseudouridine (N1mΨU), a pseudouridine (ΨU), a 5-methyluridine (5meU), and a 5-methyoxyuridine (5moU); and
(ii) a 5′ ribozyme sequence having reduced catalytic activity or a catalytically-inactive ribozyme.

57. The composition of claim 56, wherein the template RNA further comprises an unmodified uridine.

58. The composition of claim 56, wherein the template RNA is not cleavable by a ribozyme.

59. The composition of claim 56, wherein the 5′ ribozyme sequence comprises a hepatitis delta virus (HDV) ribozyme, a TriCasA ribozyme, an L8 ribozyme, or an SL28 ribozyme.

60. The composition of claim 56, wherein the 5′ ribozyme sequence comprises a sequence having at least 80% sequence identity to SEQ ID NO: 19.

61. The composition of claim 56, wherein the 5′ ribozyme sequence comprises SEQ ID NO: 19.

62. The composition of claim 56, wherein the 5′ ribozyme sequence comprises a sequence having at least 80% sequence identity to SEQ ID NO: 20.

63. The composition of claim 56, wherein the 5′ ribozyme sequence comprises SEQ ID NO: 20.

64. The composition of claim 56, wherein the 5′ ribozyme sequence comprises a complete ribozyme structure.

65. The composition of claim 56, wherein the 5′ ribozyme sequence does not comprise a complete ribozyme structure.

66. The composition of claim 56, wherein the template RNA further comprises an nrRT binding sequence that binds to the nrRT.

67. The composition of claim 66, wherein the nrRT binding sequence comprises a 3′ UTR sequence.

68. The composition of claim 67, wherein the 3′ UTR sequence is derived from an organism selected from the group consisting of Gasterosteus aculeatus, Drosophila melanogaster, Limulus polyphemus, Pungitis pungitis, Nasonia vitripennis, Geospiza fortis, Oryzias latipes, Zonotrichia albicollis, Taeniopygia guttata, Tribolium castaneum, Tinamus guttatus, Drosophila simulans, Bombyx mori, and Adineta vaga.

69. The composition of claim 67, wherein the 3′ UTR sequence comprises a sequence having at least 80% sequence identity to any one of SEQ ID NOs: 26-39.

70. The composition of claim 56, wherein the template RNA further comprises a 5′ sequence that protects a 5′ end of the template RNA from degradation.

71. The composition of claim 56, wherein the template RNA further comprises a poly A sequence.

72. The composition of claim 56, wherein the template RNA further comprises a promoter.

73. The composition of claim 56, wherein the nrRT comprises a protein domain derived from Zonotrichia albicollis, Taeniopygia guttata, Tinamus guttatus, Geospiza fortis, Pungitis pungitis, Oryzias latipes, Daniorerio, Oryzias melastigma, Petromyzon marinus, Salmo trutta, Salmo salar, Gasterosteus aculeatus, Drosophila mercatorum, Drosophila melanogaster, Nasonia vitripennis, Tribolium castaneum, Drosophila simulans, Apis cerana, Bombyx mori, Lepidurus couesii, Triops cancriformis, Limulus polyphemus, Hydra magnipapillata, Adineta vaga, or Ciona intestinalis.

74. The composition of claim 56, wherein the nrRT comprises a Taeniopygia guttata polypeptide domain.

75. The composition of claim 56, wherein the RNA encoding the nrRT comprises one or more modified uridine (U) nucleosides selected from the group consisting of a N1-methyl-pseudouridine (N1mΨU), a pseudouridine (ΨU), a 5-methyluridine (5meU), and a 5-methyoxyuridine (5moU).

76. The composition of claim 56, wherein the molar ratio of the RNA encoding the nrRT to the template RNA is from 1:1.5 to 1:1536.

77. The composition of claim 56, wherein the molar ratio of the RNA encoding the nrRT to the template RNA is 1:6.

78. A lipid nanoformulation comprising the composition of claim 56.

79. A method of modifying a eukaryotic cell, comprising transfecting the eukaryotic cell with the composition of claim 56.

80. The method of claim 79, wherein the template RNA further comprises a payload sequence, and wherein the method further comprises expressing the nrRT in the cell and inserting the payload sequence at a target site in the genome of the eukaryotic cell.

81. The method of claim 80, wherein the payload sequence is not naturally occurring in the eukaryotic cell.

82. The method of claim 80, wherein the target site comprises a 5S rDNA sequence, an 8S rDNA sequence, an 18S rDNA sequence, or a 28S rDNA sequence.

83. A composition comprising:
(a) an RNA encoding a non-LTR retrotransposon reverse transcriptase protein (nrRT) comprising a reverse transcriptase domain and an endonuclease domain; and
(b) a template RNA, wherein the template RNA further comprises one or more modified uridine (U) nucleosides selected from the group consisting of a N1-methyl-pseudouridine (N1mΨU), a pseudouridine (ΨU), a 5-methyluridine (5meU), and a 5-methyoxyuridine (5moU);
wherein the molar ratio of the RNA encoding the nrRT to the template RNA is from 1:1.5 to 1:1536.


SEQUENCE LISTING (43 sequences)

SEQ ID NO: 1 - DNA, 2235 bp, synthetic construct
  source 1..2235: mol_type = other DNA
ggataaccgcgtatgagcggtatcctggcgggagtaactatgactctcttaaggaaaagagaatcatagaacgtcagcagcctcctcgcggccccgccggtaacacagaggaacaccctgtggcgaatgctgacgatctagaaggtcgaccagatgtccgaggtcgaccagttgtccgtgtggaattgtgagcgctcacaattccacacgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacggccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccacagcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaatagcaaaaaaaaaaaaaaaaaaaaaa

SEQ ID NO: 2 - DNA, 26 bp, synthetic construct
  source 1..26: mol_type = other DNA
ggataaccgcgtatgagcggtatcct

SEQ ID NO: 3 - DNA, 116 bp, synthetic construct
  source 1..116: mol_type = other DNA
ggcgggagtaactatgactctcttaaggaaaagagaatcatagaacgtcagcagcctcctcgcggccccgccggtaacacagaggaacaccctgtggcgaatgctgacgatctaga

SEQ ID NO: 4 - DNA, 36 bp, synthetic construct
  source 1..36: mol_type = other DNA
aggtcgaccagatgtccgaggtcgaccagttgtccg

SEQ ID NO: 5 - DNA, 30 bp, synthetic construct
  source 1..30: mol_type = other DNA
tgtggaattgtgagcgctcacaattccaca

SEQ ID NO: 6 - DNA, 805 bp, synthetic construct
  source 1..805: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgtacg

SEQ ID NO: 7 - skipped

SEQ ID NO: 8 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 170 bp, synthetic construct
  source 1..170: mol_type = other DNA
aagcttgatccagacatgataagatacattgatgagtttggacaaaccacaactagaatgcagtgaaaaaaatgctttatttgtgaaatttgtgatgctattgctttatttgtaaccattataagctgcaataaacaagttgtggaattgtgagcgctcacaattccaca

SEQ ID NO: 10 - DNA, 300 bp, synthetic construct
  source 1..300: mol_type = other DNA
gcggccgctgaggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaa

SEQ ID NO: 11 - DNA, 34 bp, synthetic construct
  source 1..34: mol_type = other DNA
tagcaaaaaaaaaaaaaaaaaaaaaaaagtcttc

SEQ ID NO: 12 - DNA, 1653 bp, synthetic construct
  source 1..1653: mol_type = other DNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 13 - DNA, 239 bp, synthetic construct
  source 1..239: mol_type = other DNA
ggagacggtcaaccgcgtaggagcggtgaccggaattcggcgggagtaactatgactctcttaaggagtcatagagccagaacctcctcgtggtcccgctgggcacagggattaatttttctgtggcaaatttgactggcttcagagagcgtttttcgaagtggactgtgtgactgcgttccccccttagttgctatatccgcttcgattaacatctcacctcgacgtataagatcatt

SEQ ID NO: 14 - DNA, 134 bp, synthetic construct
  source 1..134: mol_type = other DNA
ggagaaccgcgtaggagcggtctcctggcgggagtaactatgactctcttaaaaaagagaatcatagaacgtcagcagccccctcacggccccgccggtaacacagaggaacaccctgtggcgaatgctgacga

SEQ ID NO: 15 - DNA, 131 bp, synthetic construct
  source 1..131: mol_type = other DNA
ggagaaccgcgtaggagcggtctcctggcgggagtaactatgactctcttaaaaaagagaatcatagaacgtcagcagcctcctcgcggccccgccggtaagattccgaaaggaatcgcgaatgctgacga

SEQ ID NO: 16 - DNA, 136 bp, synthetic construct
  source 1..136: mol_type = other DNA
ggagaaccgcgtaggagcggtctcctggcgggagtaactatgactctcttaaggaaaagagaatcatagaacgtcagcagcctcctcgcggccccgccggtaacacagaggaacaccctgtggcgaatgctgacga

SEQ ID NO: 17 - DNA, 114 bp, synthetic construct
  source 1..114: mol_type = other DNA
ggtaaacggcgggagtaactatgactctcttaaaaaagagaatcatagaacgtcagcggcctccacgcggccccgccggaacgcagaggaacaccctgcggcgaacgctgacgc

SEQ ID NO: 18 - DNA, 133 bp, synthetic construct
  source 1..133: mol_type = other DNA
ggataaccgcgtatgagcggtatcctggcgggagtaactatgactctcttaaaaaagagaatcatagaacgtcagcggcctccacgcggccccgccggaacgcagaggaacaccctgcggcgaacgctgacga

SEQ ID NO: 19 - DNA, 110 bp, synthetic construct
  source 1..110: mol_type = other DNA
ggcgggagtaactatgactctcttaaggaaaagagaatcatagaacgtcagcagcctcctcgcggccccgccggtaacacagaggaacaccctgtggagaatgctgacga

SEQ ID NO: 20 - DNA, 75 bp, synthetic construct
  source 1..75: mol_type = other DNA
ggaaaagagaatcatagaacgtcagcagcctcctcgcggccccgccggtaacacagaggaacaccctgtggcgaa

SEQ ID NO: 21 - DNA, 49 bp, synthetic construct
  source 1..49: mol_type = other DNA
ggcgggagtaactatgactctcttaacaaagagagaatagtaactcccg

SEQ ID NO: 22 - DNA, 26 bp, synthetic construct
  source 1..26: mol_type = other DNA
ggcgggagtaactatgactctcttaa

SEQ ID NO: 23 - DNA, 866 bp, Taeniopygia guttata
  source 1..866: mol_type = genomic DNA
ggcgggagtaactatgactctcttaagggtctagttacaactgggcatcgctgcagagatcgcacctcctcgtggtcccgctggtagcccttcgaagggtgactaagtcgatctctgccccaggtacggagccgttgggactcaccagtccaacgtaactcctgcctaaattcggtgaaacaaattcctcggtaaaaagccccatggcttcttgcccgaaacctggccccccggtttcagcaggggcaatgagtttggaaagtggactgaccacccactccgttctcgccatcgaacgtggtcccaattcgttggcaaattccggatcagactttgggggggggggtctggggctaccgttacgcctattgagggtatcggtcggcactcagacctcccgctccgactgggtagacctggtgtcctggagccacccaggacccacgtctaagtcccagcaggttgacctggtgtctttatttcctaaacaccgggttgacctgttatccaaaaacgaccaggtagacctggtggctcaatttttaccatctaaatttccccccaatttggcagaaaatgatttggctttgctggtgaacttagagttctacagatcggatttgcatgtgtatgagtgtgttcattttgctgcacattgggagggattaagtggtttgcctgaggtgtatgaacaacttgcaccacaaccgtgtgtgggagaaactttacattctagcctcccacgagacagtgaactgtttgtgcctgaagaggggagcagcgagaaggagagcgaggacgcgccaaaaacatctcctccgacgcctgggaaacatggtttggaacagactggggaggaaaaagtg

SEQ ID NO: 24 - DNA, 686 bp, Tinamus guttatus
  source 1..686: mol_type = genomic DNA
ggcgggagtaactatgactctcttaactggggaccgtggttacaacccgggcttagctgcagagacagtacctccccgtggttcccgccggaccccgtaacatcgggtgactgaatctgtctctgccccgggagtagttcctccttgccctattgaccagcggtcgccggctgctcaatagtattctaggcgtgaaatatagcgatagtcctagtggttgtcttactgggccatagccccttgcttcaggggtcattcgcgaagtctctcaggagaactgggggtggtgttcttctgggtatagctaaaccccctagactgtgtccgatccatggggtcctggatcgtgaatttcgtttcggtggcgactcagacgggagaattccctgtggatacggccaggagggcacctgtgccggtaacatcataccctgagtcggaatgccacataccgttgcccctgacattttgtaactcggatgtgactatttggggaggggttcgccctgaaccggtggactgcttgggtgatcttccagaggtgtatgatgcactcccaggggtggctgggcctcgggaatcggtgggtgggagcccgccgggagaaggggtcaggtcgccagggattgcgtcaccctctggtactgcggtccaacatgattttgggagtcccatcctcgtaccgg

SEQ ID NO: 25 - DNA, 748 bp, Zonotrichia albicollis
  source 1..748: mol_type = genomic DNA
ggcgggagtaactatgactctcttaaggcgacttgagaaggtctggttacaactgggcatagctgcagagatcgcgcctcctcgtggccccgctggtaagcccttaacagggtgactaagtcgatctctgccccagtccaggagccgctgggtttcaccagcccagcgattccttccaaattcggtgaaacaaattcctcggtaaaagccgcgtggcttattgcctgaaacctggccccccggtttcagacaggggcaaagagttcggaagtggactgaccacccaccccgaacccgagagcgaatctggtcatgacccaactgtcccaaatcctggtccgtctcttggagcgggggaaggtgcacagccactacccttactcagggtatcggtgggcacccaaacctgtgaagaggactttataacatctagaccaaccaaattacccggaattgaatcagaattaggcccgctggtgaagttttctttagaggtttacaggtcagatcttaagggggatgtgcaatttgaggggattcattttccagataattggggggtactggaggggtttcctgaggtgtacgaacaactggcaccacagccaaacgggggagacgagttaaatcatagtctcccaggggacagggagggggatgtacttgagaaggatagcagcgaaaaggagaaggaggctgcaccagaggcattgccctcagtgcaaagggcccgcagtgaacagttgcc

SEQ ID NO: 26 - DNA, 289 bp, Geospiza fortis
  source 1..289: mol_type = genomic DNA
ggtagataatctttgtatagtggggggggatctcatgtaccgggtttcttttatttgattttcaataaaacagacggtagctaggttcgcaaggcagccacaagccaaagataggtagggtgctcatagtgagtagggacagtgccttttgattcacaacgcgtcaataccatctgacacggatacccttaccggacttgtcatgatctcccagacttgtccaaggtggacgggccacctttacttaacccggaaaaggaacatatattaattatatgtgttcggaaaa

SEQ ID NO: 27 - DNA, 353 bp, Zonotrichia albicollis
  source 1..353: mol_type = genomic DNA
taggtagtcacattgcactttctgtaacttgcactgggtgtgggatgtgggcctggggtgtgggttatggggtatatatgtgggatattctggtgggaatgtccattcactgtatgcctatctttttaataaaaagacggtagctaggttcgcgaagcagccacaagccaatagccagttaggtagctcatagtgggtaggtgacaggaacctttgactcagaacgcgtccattaacatctagaacggaccaaacttcggacatgcaccgattaaccggatttgtccaaggtggacgggccacctttacttaacccggaaagggaacatatatagttatatgtgttcgtaata

SEQ ID NO: 28 - DNA, 328 bp, Taeniopygia guttata
  source 1..328: mol_type = genomic DNA
taattcaggttatttagatgcttagtttttgtacctttcttgttttgtttaggattttgatagtgttagtatttttatatttttgtacgattgcataatgttcttttttatacagttctgttttaataaaatagacgatagctagagacgttagggcagccacaagccagttaggtagcggatagtaggtaggaacagacttttactatttcataacgcgtcaattaccacctgatttggaccaattcacgggatttgtccaaggtggacgggccacctttacttaacccggaaaaggaacatatataatttatgtgtgttcgataaa

SEQ ID NO: 29 - DNA, 398 bp, Tinamus guttatus
  source 1..398: mol_type = genomic DNA
  misc_difference 100: note = a, c, t, g, unknown, or other
tagggggcttggcatttctcattgcctgctcctgaaaggatatgggtcctgcgtcgcgtggtaggcagacccattcgtccgagtagggggcttggcagtntccattgcctgtgcccgaaaggacgtgggtcatctggtctgtctgcctacacctctctagacttgtaacatctagtctgtcaacaagatcaaaattcttcacacagacgaccgagcttgctcagtcttcctgtacccgcagaattttgctcttgctctcctttggctgtgtcctggacgtgggactattccatctcgtcccaaatgccgcgtccaattataccggatttgacaaagcggacggcccgctttataagccggaaaaggtgccttgtaaaattgcaaggttcattaaatag

SEQ ID NO: 30 - DNA, 251 bp, Bombyx mori
  source 1..251: mol_type = genomic DNA
tgagccttgcacagtagtccagcggtaagggtgtagatcaggcccgtctgtttctcccccggagctcgctcccttggcttcccttatatattttaacatcagaaacagacattaaacatctactgatccaatttcgccggcgtacggccacgatcgggagggtgggaatctcgggggtcttccgatcctaatccatgatgattacgacctgagtcactaaagacgatggcatgatgatccggcgatgaaaa

SEQ ID NO: 31 - DNA, 111 bp, Oryzias latipes
  source 1..111: mol_type = genomic DNA
tgagggggacagctgggagtctcggcatgattacaaatcttgcgctgcactcggatgtcgtccccgtgacggacacattaatccggaaagcgagtggtgactcgcctcaag

SEQ ID NO: 32 - DNA, 638 bp, Tribolium castaneum
  source 1..638: mol_type = genomic DNA
taaaatctcctgaccaactagctcactgactaattttaaactgtcctgtcttacttgttttacacgtgctctgtggcggggccatttacaccccgtcgcaacacaacctgtaaatacttgtgtatgtctgtttatgtcctaatttattattttaaacagatcttggccatggtctcggccaaccaattaaagtcagtgatgcgagtcgcaatgcggagcaagagacctaggcgtgtatttattgctggcatgcggcgccggagccggtcatctgctatggggagcaatggccgggcggatacctccacgtggttccctgtgggtggcccgtcgaggacggtaaccagcgaaactccgtaaagtccttcttacgagaaggaactccggttaaagatttttccaagcctgtacacgtgattcccttggaacaagcaaagtgtggttccctcgagagggcccaggtcaggagttcgcaatagtgggctgcaagagttcatgctgggctacagtgtcaggacgaagagtgggtagtgatcgcaaaatcacgtgaatagctaccccccgcctggcaccactagacaacaacaaggggtacgacagctcttctgtcgaaagttcgggcgcacacccgtaaaagg

SEQ ID NO: 33 - DNA, 258 bp, Drosophila simulans
  source 1..258: mol_type = genomic DNA
tagctaaaacgtttggttcaaaacatttgcttgctgtcttggcataacatcaataaaggcataaacatcgcaaaataatggttatatataaatggctatgaggatggttttagtacgtaggcgttgcggaacttcggttcagatagagcaatgaatcgtgcatgctaggaaaactgaccacacgcagtgttggcagccctagtatctttcgatagatttccatacctccgcgatcaaaaaaaaaaaaaaaaaaaaaaa

SEQ ID NO: 34 - DNA, 174 bp, Pungitius pungitius
  source 1..174: mol_type = genomic DNA
tagggttcctccaccctccggctgacgaacagctggtaaacgggggggcggtggggtgcctctccagccgactgatacaggagtaagggacggtggggtcgcatccaggaagcgcagcaccgcgatgccgaaactgatgtgcagtataacacagaaagcctaaagggccaaaag

SEQ ID NO: 35 - DNA, 148 bp, Limulus polyphemus
  source 1..148: mol_type = genomic DNA
taaattttgtctctttccccaatgatgtctactagcacgctgccgaagctagatagattgaggaatctgcgtaatctgtaatgattacgcctcatgggcatctatcggtagcgtcgaccctgacgttaaattgggtaataagaaatat

SEQ ID NO: 36 - DNA, 696 bp, Nasonia vitripennis
  source 1..696: mol_type = genomic DNA
tgacctgaacaaaacgtgttgtcttgtcttgtctaaaactatttattcgaaataaggggaggctaactgcctgcaagttgaacgcgaaagttagaccttcccacctaaagcccaaaagtgatcggggaatgaatccgcgggtgaccccagagttgggtaaacccttgaaacgttggagaagcggaagagagtcccgccaccgagcatcgagtgctgcggcgcccgaatgaaaccgatcgcggatggtgcaagtcgtaggacggggcacgacctaagcctctgtcacggcggcgaagccaggaatcaccatgcaaaggtgtgaactggggcggatacctccacggggtttccctgggcatcgcgcgagcgatggccaaagtccgctttctcagctacaaaacaaaaatggtatgagacttcgttaacactaatttttccgagcctagcaggctcccttgacaacgcttatgaatctggaaaaggacacaaagtggaaaaagcgctgatggtggacaaaagtcagttgagacttgatatcagttgttttgactaagaattttattatcgttgacttttaaatattttattattgactgttaatatactgacttgggaccaagtcatctctgttacccggtaccggttcctgtcatcaaaccggaaagtccgtcccacgtaatgtggtagacgcaggag

SEQ ID NO: 37 - DNA, 1155 bp, Gasterosteus aculeatus
  source 1..1155: mol_type = genomic DNA
ggaggggagtaggtctctactctgacccgaagggcccccccgtttcagacctgattctaggctacctgtgcctaattgggggggtcccaaagagatgttgtctgttgtagaagggtttgcgccactgactgcacggaagggtgggcctcgacaggtaggggttacatgactccgtgctgctcagcagacccgcgcctctgagaccgggtagggctacttgaacaagcgacgccctggtgtatgtccgtatcctaacctggtttgggaaagccgataccggcaatgcccgccacaggtgtcgcgcaccccacgggatgacgtatgggccccgggggacctcatggatactccactggacttgcacaatcctggtgtactggatgcagcgacgttggtgacataagcaatcgctaagtcggggtaggggaggtggggacctcggcacggctgtaggaacgggtgtatgggctccggcagccgtcgtcactcccatacaacacaggggctgcatcctggtggccggtgctagttggttctggaagcccgcccgggctggttcgcagaagcagggtgcgcccagggtaggtttggtatatctgggtccggtgcgatacctatcgatgggcagcgagggccgcctcgtgacgcgctgtgtggagctggagccggcctgggtatgaacagttcttgcggatgtggcgtagctagatagtacccgtggttgtgggcgtggtgtcgaccaaatgttgtcctgtgtgcacataggccaagggttacgtgggtggcagtcagaagcacccgcacctggaagtgattgccccgggatcccggctctctgtgaagagctaccttgaggaaaggtgttccgctggaactcaagaccctacagtaggggatatcaactggctttgaggtgctgtgattccggaaccagggcgagggcgagtacttagagcatgtccaaaagcccggggaacgttccgggggcctgcttgggtcgttggacccacatccgtaaaacgatggatctcgcgtcggcgctcgggagaacttcccgcatgaacgctgattgcatgtgagaacgcccccacggcggcggggcaggcgctccccctgggtgtaaggctcgggggggtcacggctccgctctaaaag

SEQ ID NO: 38 - DNA, 256 bp, Drosophila melanogaster
  source 1..256: mol_type = genomic DNA
tagctaaatcgtttggttcaaaacatttgcttgctgtcttggcataacatcaataaaggcataaacatcgcaaaataatggttataattaaatggctatgaggatggttttagtacgtaggcgttgcggaacttcggttcatatagagcaatgaatcgtgcatgctaggaaaactgaccacacacagtgttggcagacctagtatctttcgaagatttccatacctccgcgatcaaaaaaaaaaaaaaaaaaaaaa

SEQ ID NO: 39 - DNA, 306 bp, Drosophila melanogaster
  source 1..306: mol_type = genomic DNA
tgaactagtctccttcttctattagtcagtctaattaatttttcttacattctacatctagttccattattaaattggtatgatcagtgctatctctgctacactcaatgcttaatcgtatgttattgacagtctgacacttgattactcttacgacatatgcactgtttgcttcagagaaaccactgttcatatagtgaagttcctcagttttctgttgatatattcttctttcattctcgcttctccttttctactgtgttctttttatcagttttttgtggaaaaattgagaataaataaagt

SEQ ID NO: 40 - DNA, 22 bp, synthetic construct
  source 1..22: mol_type = other DNA
ccggaatccctatctttagtcc

SEQ ID NO: 41 - DNA, 20 bp, synthetic construct
  source 1..20: mol_type = other DNA
tgctgcctttgttgctcttc

SEQ ID NO: 42 - DNA, 21 bp, synthetic construct
  source 1..21: mol_type = other DNA
aacgcgtcaataccatctgac

SEQ ID NO: 43 - DNA, 23 bp, synthetic construct
  source 1..23: mol_type = other DNA
ggaatctcgttcatccattcatg